(19) United States
(12) Patent Application Publication
(10) Pub. No.: US 2022/0387611 A1
(43) Pub. Date: Dec. 8, 2022

(54) METHODS FOR TREATING CANCER

(71) Applicant: BicycleRD Limited, Cambridge (GB)

(72) Inventors: Gavin BENNETT, Cambridge (GB); Stephen J. BLAKEMORE, Littleton, MA (US); Tara GELB, Cambridge, MA (US)

(21) Appl. No.: 17/769,709

(22) PCT Filed: Oct. 16, 2020
(86) PCT No.: PCT/GB2020/052619

§ 371 Date: Apr. 15, 2022

Related U.S. Application Data
(60) Provisional application No. 62/915,872, filed on Oct. 16, 2019.

(51) Int. Cl. A61K 47/64; A61P 35/00; G01N 33/574

(52) U.S. Cl. CPC A61K 47/64; A61P 35/00; G01N 33/57492; G01N 2333/96494

(57) ABSTRACT

The present invention relates to a method of treating cancer in a subject.

### CROSS-REFERENCE TO RELATED APPLICATIONS

The present application claims priority to U.S. provisional patent application Ser. No. 62/915,872, filed Oct. 16, 2019, the entirety of which is incorporated herein by reference.

### BACKGROUND OF THE INVENTION

MT1-MMP is a transmembrane metalloprotease that plays a major role in the extracellular matrix remodelling, directly by degrading several of its components and indirectly by activating pro-MMP2. MT1-MMP is crucial for tumor angiogenesis (Sounni et al (2002) FASEB J. 16(6), 555-564) and is over-expressed on a variety of solid tumors. Accordingly, there remains a high unmet need in developing inhibitors of MT1-MMP for the treatment of cancer.

### BRIEF DESCRIPTION OF THE DRAWING

FIG. 1 depicts body weight changes and tumor volume trace after administering BT1718 to female Balb/c nude mice bearing HT1080 xenograft. Data points represent group mean body weight and tumor volume. Error bars represent standard error of the mean (SEM).

FIG. 2 depicts tumor membrane (TM) H-scores (Y axis) and tumor stroma (TS) H-scores (X axis) for adenocarcinoma, small cell carcinoma, and squamous cell carcinoma. Squamous cell carcinoma has higher MT1-MMP membrane expression.

FIG. 3 depicts a box plot analysis of the tumor membrane (TM) H-scores for adenocarcinoma, small cell carcinoma, and squamous cell carcinoma.

### DETAILED DESCRIPTION OF CERTAIN EMBODIMENTS OF THE INVENTION

### 1. Compound

A proprietary phage display and cyclic peptide technology (Bicycle technology) was utilized to identify high affinity binding peptides to the membrane type 1-matrix metalloproteinase (MT1-MMP/MMP14). MT1-MMP (MT1) is a cell surface membrane protease normally involved in tissue remodeling which has been found to be over-expressed in many solid tumors. Overexpression of MT1 has been linked to cancer invasiveness and poor prognosis. While attempts to target the proteolytic activity of MT1 and other MMPs in cancer were unsuccessful in clinical trials largely due to toxicity caused by insufficient selectivity, MT1-MMP remains an attractive cancer target for targeted cytotoxic delivery approaches.

Diverse selection phage libraries containing 10¹¹ to 10¹³ unique peptide sequences which are post-translationally cyclized with thiol-reactive scaffolds were used to identify small (1.5-2 kDa) constrained bicyclic peptides binders (Bicycles) to the hemopexin domain of MT1. Initial binders were subject to affinity maturation by directed screens and stabilization by chemical optimization.

A bicyclic constrained peptide binder (Bicycle) was identified that binds to the hemopexin domain of MT1 with an apparent Kd of approximately 2 nM. The Bicycle peptide (N241) binds with similar affinity to the entire ectodomain of the protease but shows no binding to the catalytic domain. N241 also shows no binding toward any of the closely related MMP family members tested (MMP15, MMP16, MMP24, MMP1, Pro-MMP1, MMP2).

Characterization of the pharmacologic effect of N241 on MT1 in vitro shows that the peptide has no direct impact on the catalytic activity of the protease, nor related MMP catalytic activity (MMP1, MMP2 and MMP9) nor cell migration or invasion. However, binding of fluorescently-tagged N241 to MT1 on HT1080 fibrosarcoma cells results in the rapid internalization and subsequent lysosomal localization of the compound. In addition, ¹⁷⁷Lu-loaded N241 demonstrates rapid tumor localization when injected IV into mice bearing MT1-positive tumor xenografts, with levels as high as 15-20% injected dose per gram of tumor in less than 60 minutes. In contrast, a non-binding Bicycle peptide shows no tumor localization. These properties suggest that N241 may be a good delivery vehicle for cytotoxic payloads targeting MT1-postive tumor cells. Bicycle drug conjugates (BDCs) with a variety of linkers and cytotoxic payloads were prepared which retained binding to MT1. The anti-tumor activity of select BDCs was demonstrated in MT1-positive human tumor cell xenografts in mice.

BT1718 is a Bicycle drug conjugate (BDC) comprising a constrained bicyclic peptide that binds with high affinity and specificity to membrane type 1-matrix metalloprotease (MT1-MMP; MMP14) covalently linked through a hindered disulfide linker to the potent anti-tubulin agent DM1. MT1-MMP is naturally involved in tissue remodeling, however overexpression of the cell-surface protease has been tied to tumor aggressiveness and invasiveness, as well as poor patient prognosis for many cancer indications. The Bicycle binder for BT1718 (N241) was identified using a proprietary phage display peptide technology consisting of highly diverse phage libraries of linear amino acid sequences constrained into two loops by a central chemical scaffold. While binding with similar affinity and specificity to that observed with monoclonal antibodies, the small size of a Bicycle peptide (1.5-2 kDa) aids in its rapid extravasation and tumor penetration making it an ideal format for the targeted delivery of cytotoxic payloads.

A series of maytansinoid-BDC conjugates were prepared, with varying linker format to adjust cleavability and evaluated for their anti-tumor activity in an MT1-positive tumor xenograft model. The BDC selected for further assessment (BT1718) was evaluated for efficacy in an array of tumor xenograft models.

A mono-hindered linker-DM1 construct (BT1718) was among the most active constructs against MT1-positive EBC-1 lung tumor xenografts. Efficacy in this model was reduced in the conjugates containing the least cleavable linkers. Dosing BT1718 on a twice weekly schedule for two weeks, significant reduction in tumor growth was seen at 3 mg/kg, with 10 mg/kg causing complete regressions in this model. Effective treatment was also seen with same total dose, given at on schedules from daily to a single weekly dose. Treatment with BT1718 in a selection of MT1-positive tumor xenograft models (e.g. HT1080 fibrosarcoma; HCC1806 triple negative breast cancer; SNU-16 gastric cancer) demonstrated activity at minimally effective doses in the range of 3-10 mg/kg weekly or twice weekly, with 10 mg/kg twice weekly causing complete regressions in most models. Preliminary metabolism studies indicate that BT1718 is excreted mainly through the kidney in urine.

BT1718, a Bicycle drug conjugate (BDC), shows potent antitumor activity in human tumor xenograft models of fibrosarcoma, lung and breast cancer. Without wishing to be bound by any particular theory, it is believed that the small size of the BDC may offer a significant advantage to other targeted cytotoxic approaches such as antibody-drug conjugates due to rapid extravasation and improved tumor penetration.

In certain aspects, the present invention provides a method of treating certain cancers in a subject, comprising administering to the subject an effective amount of a drug conjugate comprising a high affinity binder of MT1-MMP, such as BT1718, or a pharmaceutically acceptable salt or composition thereof.

Preparation of BT1718 is described in detail in WO 2016/067035, filed Oct. 29, 2015, the entirety of which is hereby incorporated herein by reference. BT1718 has the structure shown below.

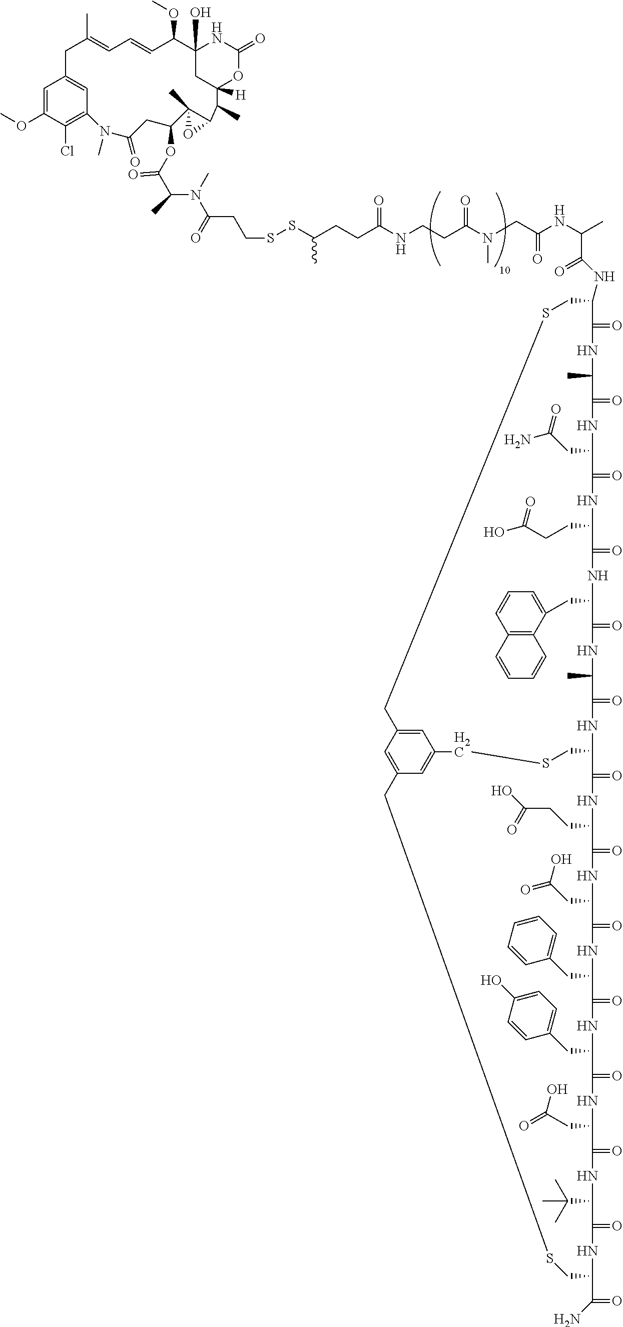

It was surprisingly found that BT1718 is highly active across tumor types shown in Table 1. In some embodiments, the cancer is selected from a tumor type in Table 1, below. In some embodiments, the tumor subtype is one of those in Table 1, below. In some embodiments, the cell line is one of those in Table 1, below.

TABLE 1
Tumor types, subtypes and representative cell lines

| Tumor | Tumor | Cell |
|---|---|---|
| Type | Subtypes | lines |
| Bladder | Basal | COV413B |
| | p53-like | ECV304 |
| | Luminal | EJ138 |
| | | HT 1197 |
| | | HT 1376 |
| | | RT112/84 |
| | | RT4 |
| | | RT4/31 |
| | | T24/83 |
| | | U-BLC1 |
| | | UM-UC-1 |
| | | UM-UC-10 |
| | | UM-UC-11 |
| | | UM-UC-13 |
| | | UM-UC-16 |
| | | UM-UC-3 |
| | | UM-UC-5 |
| | | UM-UC-6 |
| | | UM-UC-7 |
| | | UM-UC-9 |
| Endometrial | MMR-D | AN3 |
| | POLE EDM | ECC-1 |
| | p53 WT | EN |
| | p53 abnormal | EN-1 |
| | Type I | EN-11 |
| | Type II | HEC-1A |
| | carcinoma | HEC-1B |
| | carcinosarcoma | Ishikawa |
| | endometrioid adenocarcinoma | KLE |
| | serous carcinoma | MFE-280 |
| | clear cell carcinoma | MFE-296 |
| | mucinous carcinoma | MFE-319 |
| | mixed or undifferentiated | ARK1 |
| | carcinoma | ARK2 |
| | mixed serous and endometrioid | HEC-155/180 |
| | mixed serous and low-grade | SPEC-2 |
| | endometrioid | |
| | undifferentiated | |
| Esophageal | adenocarcinoma (EAC) | ESO26 |
| | squamous cell carcinoma (ESCC) | ESO51 |
| | chromosomal instability (CIN) | FLO-1 |
| | Epstein-Barr virus (EBV) | KYAE-1 |
| | genomically stable (GS) | KYSE-270 |
| | microsatellite instability (MSI) | KYSE-30 |
| | | KYSE-410 |
| | | KYSE-70 |
| | | OACM5.1 C |
| | | OE19 |
| | | OE21 |
| | | OE33 |
| | | SK-GT-2 |
| | | SK-GT-4 |
| | | OACP4 C |
| Glioblastoma | Proneural | u87-MG |
| | Neural | A 172 |
| | Classical | ANGM-CSS |
| | Mesenchymal | DBTRG.05MG |
| | | T98G |
| | | U-251 MG |
| | | U-373 MG |
| Mesothelioma | pleural mesothelioma | Mero-14 |
| | peritoneal mesothelioma | Mero-25 |
| | pericardial mesothelioma | Mero-41 |
| | epithelioid mesothelioma | Mero-48a |
| | sarcomatoid mesothelioma | Mero-82 |
| | biphasic mesothelioma | Mero-83 |
| | malignant mesothelioma | JU77 |
| | | LO68 |
| | | Mero-84 |
| | | Mero-95 |
| | | SDM103T2 |
| | | SPC111 |
| | | SPC212 |
| | | ZL34 |
| | | ZL5 |
| | | ZL55 |
| | | NO36 |
| | | ONE58 |
| Multiple | Hyperdiploid | MOLP-8 |
| Myeloma | Non-hyperdiploid | COLO 677 |
| | Cyclin D translocation | IM 9 |
| | MMSET translocation | JIM1 |
| | MAF translocation | JIM3 |
| | Unclassified | KARPAS-929 |
| | | KARPAS 417 |
| | | RPMI 8226 |
| Ovarian | clear cell | 59M |
| | endometrioid | A2780 |
| | mucinous | A2780ADR |
| | high-grade serous | A2780cis |
| | low-grade serous | Ca Ski |
| | | COLO 720 L |
| | | COV318 |
| | | COV362 |
| | | COV362.4 |
| | | COV413A |
| | | COV413B |
| | | COV434 |
| | | COV504 |
| | | COV644 |
| | | OAW28 |
| | | OAW42 |
| | | OV17R |
| | | OV56 |
| | | OV7 |
| | | PEA1 |
| | | PEA2 |
| | | PEO1 |
| | | PEO14 |
| | | PEO16 |
| | | PEO23 |
| | | PEO4 |
| | | PEO6 |
| | | SK-OV-3 |
| | | SW 626 |
| | | TO14 |
| Pancreatic | squamous | AsPC-1 |
| | pancreatic progenitor | BxPC-3 |
| | immunogenic | Capan-1 |
| | ADEX (Aberrantly Differentiated | Capan-2 |
| | Endocrine eXocrine) | CFPAC-1 |
| | | HPAC |
| | | HPAF-II |
| | | Hs 766T |
| | | MIA PaCa-2 |
| | | PANC-1 |
| | | SU.86.86 |
| Prostate | AZGP1 (subtype I) | 22Rv1 |
| | MUC1 (subtype II) | Bob |
| | MUC1 (subtype III) | LNCap clone FGC |
| | | P4E6 |
| | | PC-3 |
| | | PNT1A |
| | | PNT2 |
| | | SerBob |
| | | Shmac 1 |
| | | Shmac4 |
| | | Shmac 5 |
| | | VCaP |

It has also been found that MT1-MMP is overexpressed in endometrial cancer, ovarian cancer, bladder cancer, triple negative breast cancer, non-small cell lung cancer, and pancreatic cancer (Table 1a). Accordingly, in some embodiments, the cancer is selected from a tumor type in Table 1a, below.

TABLE 1a
MT1-MMP overexpression in certain cancers.

| | Number of | MT1-MMP |
|---|---|---|
| Tumor type | cases tested | positive* |
| Endometrial cancer | 15 | 100% |
| Ovarian cancer | 312 | 96% |
| Bladder cancer | 22 | 95% |
| Triple negative breast cancer | 41 | 76% |
| Non-small cell lung cancer | 151 | 58% |
| Pancreatic cancer | 62 | 0% |

*MT1-MMP expression was determined using IHC performed with in house validated antibody, positive cases were defined as H-score ≥50 in either tumor cell membrane or in stroma.

As defined above and described herein, the present invention provides a method of treating cancer, comprising administering to a patient in need thereof a therapeutically effective amount of BT1718, or a pharmaceutically acceptable salt and/or composition thereof. In some embodiments, the cancer is a solid tumor. In some embodiments, the cancer is associated with MT1-MMP.

In some embodiments, the cancer is high MT1-MMP expressing. For example, Adley et al. have reported that MT1-MMP has a high level of expression in clear cell carcinomas of the ovary (Adley et al. “Expression of Membrane Type 1 Matrix Metalloproteinase (MMP-14) in Epithelial Ovarian Cancer: High Level Expression in Clear Cell Carcinoma” Gynecol Oncol. 2009 February; 112(2): 319-324).

In some embodiments, the present invention provides a method of treating bladder cancer, comprising administering to a patient in need thereof a therapeutically effective amount of BT1718, or a pharmaceutically acceptable salt and/or composition thereof. In some embodiments, the present invention provides a method of treating bladder cancer, wherein the bladder cancer is selected from the group consisting of basal, p53-like, and luminal, comprising administering to a patient in need thereof a therapeutically effective amount of BT1718, or a pharmaceutically acceptable salt and/or composition thereof.

In some embodiments, the present invention provides a method of treating endometrial cancer, comprising administering to a patient in need thereof a therapeutically effective amount of BT1718, or a pharmaceutically acceptable salt and/or composition thereof. In some embodiments, the present invention provides a method of treating endometrial cancer, wherein the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated, comprising administering to a patient in need thereof a therapeutically effective amount of BT1718, or a pharmaceutically acceptable salt and/or composition thereof.

In some embodiments, the present invention provides a method of treating esophageal cancer, comprising administering to a patient in need thereof a therapeutically effective amount of BT1718, or a pharmaceutically acceptable salt and/or composition thereof. In some embodiments, the present invention provides a method of treating esophageal cancer, wherein the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI), comprising administering to a patient in need thereof a therapeutically effective amount of BT1718, or a pharmaceutically acceptable salt and/or composition thereof.

In some embodiments, the present invention provides a method of treating lung cancer, comprising administering to a patient in need thereof a therapeutically effective amount of BT1718, or a pharmaceutically acceptable salt and/or composition thereof. In some embodiments, the present invention provides a method of treating lung cancer, wherein the lung cancer is selected from the group consisting of NSCLC adenocarcinoma, small cell carcinoma, and NSCLC squamous cell carcinoma, comprising administering to a patient in need thereof a therapeutically effective amount of BT1718, or a pharmaceutically acceptable salt and/or composition thereof.

In some embodiments, the present invention provides a method of treating glioblastoma, comprising administering to a patient in need thereof a therapeutically effective amount of BT1718, or a pharmaceutically acceptable salt and/or composition thereof. In some embodiments, the present invention provides a method of treating glioblastoma, wherein the glioblastoma is selected from the group consisting of proneural, neural, classical, and mesenchymal, comprising administering to a patient in need thereof a therapeutically effective amount of BT1718, or a pharmaceutically acceptable salt and/or composition thereof.

In some embodiments, the present invention provides a method of treating a mesothelioma, comprising administering to a patient in need thereof a therapeutically effective amount of BT1718, or a pharmaceutically acceptable salt and/or composition thereof. In some embodiments, the present invention provides a method of treating mesothelioma, wherein the mesothelioma is selected from the group consisting of pleural mesothelioma, peritoneal mesothelioma, pericardial mesothelioma, epithelioid mesothelioma, sarcomatoid mesothelioma, biphasic mesothelioma, and malignant mesothelioma, comprising administering to a patient in need thereof a therapeutically effective amount of BT1718, or a pharmaceutically acceptable salt and/or composition thereof.

In some embodiments, the present invention provides a method of treating multiple myeloma, comprising administering to a patient in need thereof a therapeutically effective amount of BT1718, or a pharmaceutically acceptable salt and/or composition thereof. In some embodiments, the present invention provides a method of treating multiple myeloma, wherein the multiple myeloma is selected from the group consisting of hyperdiploid, non-hyperdiploid, cyclin D translocation, MMSET translocation, MAF translocation, and unclassified, comprising administering to a patient in need thereof a therapeutically effective amount of BT1718, or a pharmaceutically acceptable salt and/or composition thereof.

In some embodiments, the present invention provides a method of treating ovarian cancer, comprising administering to a patient in need thereof a therapeutically effective amount of BT1718, or a pharmaceutically acceptable salt and/or composition thereof. In some embodiments, the present invention provides a method of treating ovarian cancer, wherein the ovarian cancer is selected from the group consisting of clear cell, endometrioid, mucinous, high-grade serous and low-grade serous ovarian cancer, comprising administering to a patient in need thereof a therapeutically effective amount of BT1718, or a pharmaceutically acceptable salt and/or composition thereof.

In some embodiments, the present invention provides a method of treating pancreatic cancer, comprising administering to a patient in need thereof a therapeutically effective amount of BT1718, or a pharmaceutically acceptable salt and/or composition thereof. In some embodiments, the present invention provides a method of treating pancreatic cancer, wherein the pancreatic cancer is selected from the group consisting of squamous, pancreatic progenitor, immunogenic, and ADEX (Aberrantly Differentiated Endocrine eXocrine) pancreatic cancer, comprising administering to a patient in need thereof a therapeutically effective amount of BT1718, or a pharmaceutically acceptable salt and/or composition thereof.

In some embodiments, the present invention provides a method of treating prostate cancer, comprising administering to a patient in need thereof a therapeutically effective amount of BT1718, or a pharmaceutically acceptable salt and/or composition thereof. In some embodiments, the present invention provides a method of treating prostate cancer, wherein the prostate cancer is selected from the group consisting of AZGP1 (subtype I), MUC1 (subtype II), and MUC1 (subtype III) prostate cancer, comprising administering to a patient in need thereof a therapeutically effective amount of BT1718, or a pharmaceutically acceptable salt and/or composition thereof.

In some embodiments, the amount administered to the patient of BT1718, or a pharmaceutically acceptable salt thereof, is a minimally effective dose that is <3 mg/kg, wherein the minimally effective dose leads to tumor-stasis when administered intravenous twice a week (IV BIW), or a pharmaceutically acceptable salt thereof. In some embodiments, the amount administered to the patient of BT1718, or a pharmaceutically acceptable salt and/or composition thereof, is a minimally effective dose that is <10 mg/kg, wherein the minimally effective dose leads to tumor-stasis when administered IV BIW, or a pharmaceutically acceptable salt and/or composition thereof. In some embodiments, the amount administered to the patient of BT1718, or a pharmaceutically acceptable salt and/or composition thereof, is a minimally effective dose that is 3 mg/kg, wherein the minimally effective dose leads to tumor-stasis when administered IV BIW, or a pharmaceutically acceptable salt and/or composition thereof. In some embodiments, the amount administered to the patient of BT1718, or a pharmaceutically acceptable salt and/or composition thereof, is a minimally effective dose that is 10 mg/kg, wherein the minimally effective dose leads to tumor-stasis when administered IV BIW, or a pharmaceutically acceptable salt and/or composition thereof.

In some embodiments, the invention provides a method of identifying or selecting a patient having an elevated MT1-MMP level in a tumor tissue, comprising measuring staining intensity in a tumor tissue section of a patient using an MT1-MMP IHC staining assay, and selecting a patient who is staining positive in the MT1-MMP IHC staining assay. In some embodiments, an MT1-MMP IHC staining assay is as described in the examples of the instant application.

As used herein, the term “an elevated MT1-MMP level” refers to that certain percentage of cells in a tumor tissue have a detectable amount of MT1-MMP, for example, on tumor cell membrane, or in tumor stroma, or both. In some embodiments, MT1-MMP positive refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of cells in a tumor tissue have a detectable amount of MT1-MMP, for example, on tumor cell membrane, or in tumor stroma, or both.

As used herein, the term “a patient who is staining positive” refers to a patient having certain percentage of cells in a tumor tissue section which are staining positive in a MT1-MMP IHC staining assay. In some embodiments, a patient who is staining positive has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of cells in a tumor tissue section which are staining positive in an MT1-MMP IHC staining assay.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

There are a variety of methods to measure staining intensity in an IHC staining assay. In some embodiments, staining intensity is measured by visual scoring, for example, by manual scoring using conventional light microscopy. In some embodiments, staining intensity is measured by computational tissue analysis (CTA) scoring. The staining intensity levels can be no staining (0), weak staining (1+), median staining (2+), or strong staining (3+). In some embodiments, a cell which is staining positive refers to a cell with a stain intensity of weak staining (1+), median staining (2+), or strong staining (3+). In some embodiments, a cell which is staining positive refers to a cell with a stain intensity of median staining (2+) or strong staining (3+). In some embodiments, a cell which is staining positive refers to a stain intensity of strong staining (3+). In some embodiments, staining intensity is measured on tumor cell membrane of a tumor tissue section. In some embodiments, staining intensity is measured on tumor cell cytoplasm of a tumor tissue section. In some embodiments, staining intensity is measured in cells present in tumor stroma of a tumor tissue section. In some embodiments, staining intensity is measured both on tumor cell membrane and in cells present in tumor stroma of a tumor tissue section.

In some embodiments, a patient who is staining positive refers to a patient having an H-score of about 15 or more in a tumor tissue section in an IHC staining assay. In some embodiments, a patient who is staining positive refers to a patient having an H-score of about 20 or more in a tumor tissue section in an IHC staining assay. In some embodiments, a patient who is staining positive refers to a patient having an H-score of about 30 or more in a tumor tissue section in an IHC staining assay. In some embodiments, a patient who is staining positive refers to a patient having an H-score of about 40 or more in a tumor tissue section in an IHC staining assay. In some embodiments, a patient who is staining positive refers to a patient having an H-score of about 50 or more in a tumor tissue section in an IHC staining assay. In some embodiments, a patient who is staining positive refers to a patient having an H-score of about 75 or more in a tumor tissue section in an IHC staining assay. In some embodiments, a patient who is staining positive refers to a patient having an H-score of about 100 or more in a tumor tissue section in an IHC staining assay. In some embodiments, a patient who is staining positive refers to a patient having an H-score of about 125 or more in a tumor tissue section in an IHC staining assay. In some embodiments, a patient who is staining positive refers to a patient having an H-score of about 150 or more in a tumor tissue section in an IHC staining assay. In some embodiments, a patient who is staining positive refers to a patient having an H-score of about 200 or more in a tumor tissue section in an IHC staining assay. In some embodiments, a patient who is staining positive refers to a patient having an H-score of about 250 or more in a tumor tissue section in an IHC staining assay. In some embodiments, a patient who is staining positive refers to a patient having an H-score of about 300 or more in a tumor tissue section in an IHC staining assay.

An H-score is the sum of the products of the percent of cells×their staining intensity on a scale of 0-3 as described above (no staining (0), weak staining (1+), median staining (2+), or strong staining (3+)):

[((0×(% cells at 0))+((1×(% cells at 1+))+((2×(% cells at 2+))+((3×(% cells at 3))]

An H-score can be generated for different compartment in a tumor tissue section, including, for example, the tumor cell membrane and tumor stroma. In some embodiments, an H-score refers to an H-score for tumor cell membrane, which is the sum of the products of the percent of tumor cells with positive membrane staining×their cell membrane staining intensity on a scale of 0-3 as described above. In some embodiments, an H-score refers to an H-score for cells present in tumor stroma, which is the sum of the products of the percent of cells present in the tumor stroma with positive staining×their stromal staining intensity on a scale of 0-3 as described above.

In some embodiments, a patient who is staining positive refers to a patient having an H-score for tumor cell membrane of about 15 or more in a tumor tissue section in an IHC staining assay. In some embodiments, a patient who is staining positive refers to a patient having an H-score for tumor cell membrane of about 20 or more in a tumor tissue section in an IHC staining assay. In some embodiments, a patient who is staining positive refers to a patient having an H-score for tumor cell membrane of about 30 or more in a tumor tissue section in an IHC staining assay. In some embodiments, a patient who is staining positive refers to a patient having an H-score for tumor cell membrane of about 40 or more in a tumor tissue section in an IHC staining assay. In some embodiments, a patient who is staining positive refers to a patient having an H-score for tumor cell membrane of about 50 or more in a tumor tissue section in an IHC staining assay. In some embodiments, a patient who is staining positive refers to a patient having an H-score for tumor cell membrane of about 75 or more in a tumor tissue section in an IHC staining assay. In some embodiments, a patient who is staining positive refers to a patient having an H-score for tumor cell membrane of about 100 or more in a tumor tissue section in an IHC staining assay. In some embodiments, a patient who is staining positive refers to a patient having an H-score for tumor cell membrane of about 125 or more in a tumor tissue section in an IHC staining assay. In some embodiments, a patient who is staining positive refers to a patient having an H-score for tumor cell membrane of about 150 or more in a tumor tissue section in an IHC staining assay. In some embodiments, a patient who is staining positive refers to a patient having an H-score for tumor cell membrane of about 200 or more in a tumor tissue section in an IHC staining assay. In some embodiments, a patient who is staining positive refers to a patient having an H-score for tumor cell membrane of about 250 or more in a tumor tissue section in an IHC staining assay. In some embodiments, a patient who is staining positive refers to a patient having an H-score for tumor cell membrane of about 300 or more in a tumor tissue section in an IHC staining assay.

In some embodiments, a patient who is staining positive refers to a patient having an H-score for cells present in tumor stroma of about 15 or more in a tumor tissue section in an IHC staining assay. In some embodiments, a patient who is staining positive refers to a patient having an H-score for cells present in tumor stroma of about 20 or more in a tumor tissue section in an IHC staining assay. In some embodiments, a patient who is staining positive refers to a patient having an H-score for cells present in tumor stroma of about 30 or more in a tumor tissue section in an IHC staining assay. In some embodiments, a patient who is staining positive refers to a patient having an H-score for cells present in tumor stroma of about 40 or more in a tumor tissue section in an IHC staining assay. In some embodiments, a patient who is staining positive refers to a patient having an H-score for cells present in tumor stroma of about 50 or more in a tumor tissue section in an IHC staining assay. In some embodiments, a patient who is staining positive refers to a patient having an H-score for cells present in tumor stroma of about 75 or more in a tumor tissue section in an IHC staining assay. In some embodiments, a patient who is staining positive refers to a patient having an H-score for cells present in tumor stroma of about 100 or more in a tumor tissue section in an IHC staining assay. In some embodiments, a patient who is staining positive refers to a patient having an H-score for cells present in tumor stroma of about 125 or more in a tumor tissue section in an IHC staining assay. In some embodiments, a patient who is staining positive refers to a patient having an H-score for cells present in tumor stroma of about 150 or more in a tumor tissue section in an IHC staining assay. In some embodiments, a patient who is staining positive refers to a patient having an H-score for cells present in tumor stroma of about 200 or more in a tumor tissue section in an IHC staining assay. In some embodiments, a patient who is staining positive refers to a patient having an H-score for cells present in tumor stroma of about 250 or more in a tumor tissue section in an IHC staining assay. In some embodiments, a patient who is staining positive refers to a patient having an H-score for cells present in tumor stroma of about 300 or more in a tumor tissue section in an IHC staining assay.

In some embodiments, a patient who is staining positive refers to a patient having a composite H-score (for tumor cell membrane and cells present in tumor stroma) of about 15 or more in a tumor tissue section in an IHC staining assay. In some embodiments, a patient who is staining positive refers to a patient having a composite H-score (for tumor cell membrane and cells present in tumor stroma) of about 20 or more in a tumor tissue section in an IHC staining assay. In some embodiments, a patient who is staining positive refers to a patient having a composite H-score (for tumor cell membrane and cells present in tumor stroma) of about 30 or more in a tumor tissue section in an IHC staining assay. In some embodiments, a patient who is staining positive refers to a patient having a composite H-score (for tumor cell membrane and cells present in tumor stroma) of about 40 or more in a tumor tissue section in an IHC staining assay. In some embodiments, a patient who is staining positive refers to a patient having a composite H-score (for tumor cell membrane and cells present in tumor stroma) of about 50 or more in a tumor tissue section in an IHC staining assay. In some embodiments, a patient who is staining positive refers to a patient having a composite H-score (for tumor cell membrane and cells present in tumor stroma) of about 75 or more in a tumor tissue section in an IHC staining assay. In some embodiments, a patient who is staining positive refers to a patient having a composite H-score (for tumor cell membrane and cells present in tumor stroma) of about 100 or more in a tumor tissue section in an IHC staining assay. In some embodiments, a patient who is staining positive refers to a patient having a composite H-score (for tumor cell membrane and cells present in tumor stroma) of about 125 or more in a tumor tissue section in an IHC staining assay. In some embodiments, a patient who is staining positive refers to a patient having a composite H-score (for tumor cell membrane and cells present in tumor stroma) of about 150 or more in a tumor tissue section in an IHC staining assay. In some embodiments, a patient who is staining positive refers to a patient having a composite H-score (for tumor cell membrane and cells present in tumor stroma) of about 200 or more in a tumor tissue section in an IHC staining assay. In some embodiments, a patient who is staining positive refers to a patient having a composite H-score (for tumor cell membrane and cells present in tumor stroma) of about 250 or more in a tumor tissue section in an IHC staining assay. In some embodiments, a patient who is staining positive refers to a patient having a composite H-score (for tumor cell membrane and cells present in tumor stroma) of about 300 or more in a tumor tissue section in an IHC staining assay. In some embodiments, a patient who is staining positive refers to a patient having a composite H-score (for tumor cell membrane and cells present in tumor stroma) of about 400 or more in a tumor tissue section in an IHC staining assay. In some embodiments, a patient who is staining positive refers to a patient having a composite H-score (for tumor cell membrane and cells present in tumor stroma) of about 500 or more in a tumor tissue section in an IHC staining assay.

A composite H-score (for tumor cell membrane and cells present in tumor stroma) is determined by combining the individually determined H-scores for tumor cell membrane and for cells present in tumor stroma. In some embodiments, the composite H-score is the sum of the individually determined H-scores for tumor cell membrane and for cells present in tumor stroma. In some embodiments, the composite H-score is a weighted sum of the individually determined H-scores for tumor cell membrane and for cells present in tumor stroma.

In some embodiments, the invention provides a method of identifying or selecting a patient having an elevated MT1-MMP level in a tumor tissue, comprising measuring staining intensity in a tumor tissue section of a patient using an MT1-MMP IHC staining assay, and selecting a patient having a composite H-score (for tumor cell membrane and cells present in tumor stroma) of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more.

In some embodiments, the invention provides a method of identifying or selecting a patient having an elevated MT1-MMP level in a tumor tissue, comprising measuring staining intensity in a tumor tissue section of a patient using an MT1-MMP IHC staining assay, and selecting a patient having an H-score for tumor cell membrane of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more.

In some embodiments, the invention provides a method of identifying or selecting a patient having an elevated MT1-MMP level in a tumor tissue, comprising measuring staining intensity in a tumor tissue section of a patient using an MT1-MMP IHC staining assay, and selecting a patient having an H-score for cells present in tumor stroma of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more.

In some embodiments, the present invention provides a method of treating a cancer in a patient having an elevated MT1-MMP level in a tumor tissue, comprising administering to a patient in need thereof a therapeutically effective amount of BT1718, or a pharmaceutically acceptable salt thereof, or a pharmaceutical composition thereof. In some embodiments, a patient having an elevated MT1-MMP level is selected according to a method as described herein.

In some embodiments, the present invention provides a method of treating a cancer in a patient, comprising selecting a patient having an elevated MT1-MMP level in a tumor tissue, and administering to a patient in need thereof a therapeutically effective amount of BT1718, or a pharmaceutically acceptable salt thereof, or a pharmaceutical composition thereof. In some embodiments, selecting a patient having an elevated MT1-MMP level is as described in a method provided herein.

In some embodiments, the present invention provides a method of treating a cancer in a patient, comprising measuring MT1-MMP level in a tumor tissue section of a patient using an MT1-MMP IHC staining assay, selecting a patient having an elevated MT1-MMP level in a tumor tissue (for example, as described in a method provided herein), and administering to a patient in need thereof a therapeutically effective amount of BT1718, or a pharmaceutically acceptable salt thereof, or a pharmaceutical composition thereof. In some embodiments, measuring MT1-MMP level in a tumor tissue section of a patient using an MT1-MMP IHC staining assay is as described in a method provided herein.

In some embodiments, a cancer is pancreatic cancer. In some embodiments, a cancer is stomach cancer. In some embodiments, a cancer is bladder cancer. In some embodiments, a cancer is head & neck cancer. In some embodiments, a cancer is lung cancer. In some embodiments, a cancer is a triple negative breast cancer (TNBC). In some embodiments, a cancer is ovarian cancer.

In some embodiments, the lung cancer is non-small cell lung cancer (NSCLC). In some embodiments, the lung cancer is small cell lung cancer (SCLC).

In some embodiments, the present invention provides a method of treating a cancer in a patient having a composite H-score of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more in a tumor tissue section in an MT1-MMP IHC staining assay, comprising administering to a patient in need thereof a therapeutically effective amount of BT1718, or a pharmaceutically acceptable salt thereof, or a pharmaceutical composition thereof. In some embodiments, an MT1-MMP IHC staining assay is as described herein.

In some embodiments, the present invention provides a method of treating a cancer in a patient having an H-score for tumor cell membrane of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more in a tumor tissue section in an MT1-MMP IHC staining assay, comprising administering to a patient in need thereof a therapeutically effective amount of BT1718, or a pharmaceutically acceptable salt thereof, or a pharmaceutical composition thereof.

In some embodiments, the present invention provides a method of treating a cancer in a patient having an H-score for cells present in tumor stroma of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more in a tumor tissue section in an MT1-MMP IHC staining assay, comprising administering to a patient in need thereof a therapeutically effective amount of BT1718, or a pharmaceutically acceptable salt thereof, or a pharmaceutical composition thereof.

In some embodiments, the present invention provides a method of treating a cancer in a patient, comprising selecting a patient having a composite H-score of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more in a tumor tissue section in an MT1-MMP IHC staining assay, and administering to a patient in need thereof a therapeutically effective amount of BT1718, or a pharmaceutically acceptable salt thereof, or a pharmaceutical composition thereof.

In some embodiments, the present invention provides a method of treating a cancer in a patient, comprising selecting a patient having an H-score for tumor cell membrane of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more in a tumor tissue section in an MT1-MMP IHC staining assay, and administering to a patient in need thereof a therapeutically effective amount of BT1718, or a pharmaceutically acceptable salt thereof, or a pharmaceutical composition thereof.

In some embodiments, the present invention provides a method of treating a cancer in a patient, comprising selecting a patient having an H-score for cells present in tumor stroma of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more in a tumor tissue section in an MT1-MMP IHC staining assay, and administering to a patient in need thereof a therapeutically effective amount of BT1718, or a pharmaceutically acceptable salt thereof, or a pharmaceutical composition thereof.

In some embodiments, the present invention provides a method of treating a cancer in a patient, comprising measuring staining intensity in a tumor tissue section of a patient using an MT1-MMP IHC staining assay, selecting a patient having a composite H-score of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more, and administering to a patient in need thereof a therapeutically effective amount of BT1718, or a pharmaceutically acceptable salt thereof, or a pharmaceutical composition thereof.

In some embodiments, the present invention provides a method of treating a cancer in a patient, comprising measuring staining intensity in a tumor tissue section of a patient using an MT1-MMP IHC staining assay, selecting a patient having an H-score for tumor cell membrane of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more, and administering to a patient in need thereof a therapeutically effective amount of BT1718, or a pharmaceutically acceptable salt thereof, or a pharmaceutical composition thereof.

In some embodiments, the present invention provides a method of treating a cancer in a patient, comprising measuring staining intensity in a tumor tissue section of a patient using an MT1-MMP IHC staining assay, selecting a patient having an H-score for cells present in tumor stroma of about 15 or more, about 20 or more, about 30 or more, about 40 or more, about 50 or more, about 75 or more, about 100 or more, about 125 or more, or about 150 or more, or about 200 or more, or about 250 or more, or about 300 or more, or about 400 or more, or about 500 or more, and administering to a patient in need thereof a therapeutically effective amount of BT1718, or a pharmaceutically acceptable salt thereof, or a pharmaceutical composition thereof.

Pharmaceutically Acceptable Compositions

According to another embodiment, the invention provides a composition comprising BT1718, or a pharmaceutically acceptable salt thereof, and a pharmaceutically acceptable carrier, adjuvant, or vehicle.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “subject,” as used herein, is used interchangeably with the term “patient” and means an animal, preferably a mammal. In some embodiments, a subject or patient is a human. In other embodiments, a subject (or patient) is a veterinary subject (or patient). In some embodiments, a veterinary subject (or patient) is a canine, a feline, or an equine subject.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of BT1718, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

BT1718, or a pharmaceutically acceptable salt thereof, can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXEMPLIFICATION

The following Examples illustrate the invention described above; they are not, however, intended to limit the scope of the invention in any way. The beneficial effects of the pharmaceutical compounds, combinations, and compositions of the present invention can also be determined by other test models known as such to the person skilled in the pertinent art.

### Example 1

### Evaluation of the Efficacy of BT1718 in a Xenograft Model in Female BALB/C Nude Mice

### Study Objective

The objective of this study is to evaluate the anti-tumor efficacy of BT1718 in a xenograft model of bladder, endometrial, esophageal, glioblastoma, mesothelioma, multiple myeloma, ovarian, pancreatic, and/or prostate cancer in female BALB/c nude mice.

TABLE 2
Experimental Design

| | | | Dose | Dose | Conc. | Dosing | |
|---|---|---|---|---|---|---|---|
| Gr | n | Treatment | (mg/kg) | volume(ml/kg) | (mg/ml) | Route | Schedule* |
| 1 | 3 | Vehicle | — | 10 | — | IV | tiw*2 |
| | | | | | | | weeks |
| 2 | 3 | BT1718 | 30 | 10 | 3 | IV | qw*2 |
| | | | | | | | weeks |
| 3 | 3 | BT1718 | 15 | 10 | 1.5 | IV | biw*2 |
| | | | | | | | weeks |
| 4 | 3 | BT1718 | 10 | 10 | 1 | IV | tiw*2 |
| | | | | | | | weeks |
| 5 | 3 | BT1718 | 4.3 | 10 | 0.43 | IV | qd*2 |
| | | | | | | | weeks |
| 6 | 3 | BT1718 | 9 | 10 | 0.9 | IV | qw*2 |
| | | | | | | | weeks |
| 7 | 3 | BT1718 | 4.5 | 10 | 0.45 | IV | biw*2 |
| | | | | | | | weeks |
| 8 | 3 | BT1718 | 3 | 10 | 0.3 | IV | tiw*2 |
| | | | | | | | weeks |
| 9 | 3 | BT1718 | 1.3 | 10 | 0.13 | IV | qd*2 |
| | | | | | | | weeks |

Note:
n: animal number; Dosing volume: adjust dosing volume based on body weight 10 μl/g;
*Schedule: tiw = 3 times per week; qw = 1 time per week; biw = 2 times per week; qd = once a day.

### Materials

### Animals and Housing Condition

Animals

Species: Mus Musculus

Strain: Balb/c nude

Age: 6-8 weeks

Sex: female

Body weight: 18-22 g

Number of animals: 27 mice plus spare

### Housing Condition

The mice are kept in individual ventilation cages at constant temperature and humidity with 3 animals in each cage.

Temperature: 2026° C.

Humidity 40-70%.

Cages: Made of polycarbonate. The size is 300 mm×180 mm×150 mm. The bedding material is corn cob, which is changed twice per week.

Diet: Animals have free access to irradiation sterilized dry granule food during the entire study period.

Water: Animals have free access to sterile drinking water.

Cage identification: The identification labels for each cage contain the following information: number of animals, sex, strain, date received, treatment, study number, group number and the starting date of the treatment.

Animal identification: Animals are marked by ear coding.

### Test and Positive Control Articles

Product identification: BT1718

Manufacturer: Bicycle Therapeutics

Lot number: N/A

Physical description: Clear solution (in DMSO)

Molecular weight: 3511.4, Formula weight: 3511.4, Purity: >95%

Package and storage condition: store at −80° C.

### Experimental Methods and Procedures

### Cell Culture

The appropriate tumor cells are maintained in vitro as a monolayer culture in a suitable medium at 37° C. in an atmosphere of 5% CO₂ in air. The tumor cells are routinely subcultured twice weekly by trypsin-EDTA treatment. The cells growing in an exponential growth phase are harvested and counted for tumor inoculation.

### Tumor Inoculation

Each mouse is inoculated subcutaneously at the right flank with tumor cells (10×10⁶) in 0.2 ml of PBS for tumor development. The animals are randomized and treatment is started when the average tumor volume reaches approximately 165 mm³ for the efficacy study. The test article administration and the animal numbers in each group are shown in Table 2.

### Observations

All the procedures related to animal handling, care and the treatment in the study are performed according to the guidelines of the Association for Assessment and Accreditation of Laboratory Animal Care (AAALAC). At the time of routine monitoring, the animals are daily checked for any effects of tumor growth and treatments on normal behavior such as mobility, food and water consumption (by looking only), body weight gain/loss (body weights are measured every day), eye/hair matting and any other abnormal effect as stated in the protocol. Death and observed clinical signs are recorded on the basis of the numbers of animals within each subset.

### Tumor Measurements and the Endpoints

The major endpoint is to see if the tumor growth could be delayed or mice could be cured. Tumor size is measured three times weekly in two dimensions using a caliper, and the volume is expressed in mm³ using the formula: V=0.5 a×b² where a and b are the long and short diameters of the tumor, respectively. The tumor size is then used for calculations of T/C value. The T/C value (in percent) is an indication of antitumor effectiveness; T and C are the mean volumes of the treated and control groups, respectively, on a given day.

TGI is calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

### Statistical Analysis

Summary statistics, including mean and the standard error of the mean (SEM), is provided for the tumor volume of each group at each time point.

Statistical analysis of difference in tumor volume among the groups is conducted on the data obtained at the best therapeutic time point after the final dose.

A one-way ANOVA is performed to compare tumor volume among groups, and when a significant F-statistics (a ratio of treatment variance to the error variance) is obtained, comparisons between groups are carried out with Games-Howell test. All data are analyzed using Prism. P<0.05 is considered to be statistically significant.

### Results

### Mortality, Morbidity, and Body Weight Gain or Loss

Animal body weight is monitored regularly as an indirect measure of toxicity.

### Tumor Volume Trace

Mean tumor volume over time in female Balb/c nude mice bearing cancer cell xenograft is monitored.

### Tumor Growth Inhibition Analysis

Tumor growth inhibition rate for BT1718 in the xenograft model is calculated based on tumor volume measurements at day 14 after the start of treatment. Tumor Growth Inhibition is calculated by dividing the group average tumor volume for the treated group by the group average tumor volume for the control group (T/C). For a test article to be considered to have anti-tumor activity, T/C must be 50% or less.

### Example 2

### Evaluation of the Efficacy of BB1718 in a Patient-Derived Xenograft (PDX) Model in Female Balb/C Nude Mice

### Study Objective

The objective of the project is to evaluate the in vivo therapeutic efficacy of test articles in a PDX model of bladder, endometrial, esophageal, glioblastoma, mesothelioma, multiple myeloma, ovarian, pancreatic, and/or prostate cancer in female BALB/c nude mice. The bladder, endometrial, esophageal, glioblastoma, mesothelioma, multiple myeloma, ovarian, pancreatic, and/or prostate cancer cell line used is high MT1-expressing.

### Study Design

The PDX model is shown in Table 3.

TABLE 3
PDX model used in study

| Model | | | MT1-MMP |
|---|---|---|---|
| Name | Cancer Type | Tumor growth speed | expression |
| PDX | bladder, endometrial, | Tumor size can | High |
| | esophageal, glioblastoma, | reach 1000 mm³ | |
| | mesothelioma, multiple | in 40 days after | |
| | myeloma, ovarian, | tumor inoculation | |
| | pancreatic, and/or | | |
| | prostate cancer | | |

### Animals

Balb/C nude, female, 6-8 weeks, weighing approximately 18-22 g.

### Tumor Inoculation

Each mouse is inoculated subcutaneously at the right flank with a certain kind of tumor fragment (30 mm³) for tumor development. The treatments start when the average tumor volume reaches approximately 150-200 mm³. The test article administration and the animal numbers in each group are shown in Table 4 which details the experimental design.

TABLE 4
Study Design

| | | | | Dosing | | |
|---|---|---|---|---|---|---|
| | | | Dose | Volume | Dosing | |
| Group | Treatment | n | (mg/kg) | (μL/g) | Route | Schedule |
| 1 | Vehicle | 6 | — | 10 | iv | biw × |
| | | | | | | 4 wks |
| 2 | BT1718 | 6 | 3 | 10 | iv | biw × |
| | | | | | | 4 wks |
| 3 | BT1718 | 6 | 10 | 10 | iv | biw × |
| | | | | | | 4 wks |
| 4 | Comparator | 6 | | | iv | biw × |
| | | | | | | 4 wks |

Treatment is given until the control tumors reach an average of 1000 mm³.

### Animal Housing

An acclimation period of approximately one week is allowed between animal arrival and tumor inoculation in order to accustom the animals to the laboratory environment. The mice are maintained in a special pathogen-free environment and in individual ventilation cages (3 mice per cage). All cages, bedding, and water are sterilized before use when working in the mouse room, the investigators wear lab coats and latex or vinyl gloves. Each cage is clearly labeled with a cage card indicating number of animals, sex, strain, date received, treatment, study number, group number and the starting date of the treatment The cages with food and water are changed twice a week. The targeted conditions for animal room environment and photoperiod are as follows:

Temperature: 20-26° C.

Humidity: 40-70%

Light cycle: 12 hours light and 12 hours dark

### Dietary Materials

All animals have free access to a standard certified commercial laboratory diet. Maximum allowable concentrations of contaminants in the diet are controlled and routinely analyzed by the manufacturers. Autoclaved municipal tap water suitable for human consumption is available to the animals ad libitum. It is considered that there are no known contaminants in the dietary materials that could influence the tumor growth

### Assignment to Groups

Before commencement of treatment all animals are weighed and the tumor volumes are measured. Since the tumor volume can affect the effectiveness of any given treatment, mice are assigned into groups using randomized block design based upon their tumor volumes. This ensures that all the groups are comparable at the baseline.

### Observations

The protocol and any amendment(s) or procedures involving the care and use of animals in this study are reviewed and approved prior to conduct. During the study, the care and use of animals is conducted in accordance with the regulations of the Association for Assessment and Accreditation of Laboratory Animal Care (AAALAC). After inoculation, the animals are checked daily for morbidity and mortality. At the time of routine monitoring, the animals are checked for any effects of tumor growth and treatments on normal behavior such as mobility, food and water consumption, body weight gain/loss (body weights will be measured twice weekly), eye/hair matting and any other abnormal effect. Death and observed clinical signs are recorded on the basis of the numbers of animals within each subset.

### Endpoints

The major endpoint is to see if the tumor growth can be delayed or mice can be cured. Tumor sizes are measured twice weekly in two dimensions using a caliper and the volume is expressed in mm³ using the formula: V=0.5 a×b² where a and b are the long and short diameters of the tumor, respectively. The tumor sizes are used for the calculations of both T-C and TIC values. T-C is calculated with T as the median time (in days) required for the treatment group tumors to reach a predetermined size (e.g., 1.000 mm³), and C is the median time (in days) for the control group tumors to reach the same size. The TIC value (in percent) is an indication of antitumor effectiveness, T and C are the mean volume of the treated and control groups, respectively, on a given day.

TGI is calculated for each group using the formula. TGI (%)=[1−(Ti−T0)/(Vi−V0)]×100; Ti is the average tumor volume of a treatment group on a given day. T0 is the average tumor volume of the treatment group on the first day of treatment. Vi is the average tumor volume of the vehicle control group on the same day with Ti, and V0 is the average tumor volume of the vehicle group on the first day of treatment.

### Statistical Analysis

For comparison between two groups, an independent sample t-test is used for comparison among three or more groups, a one-way ANOVA will be performed if a significant F-statistics (a ratio of treatment variance to the error variance) is obtained, multiple comparison procedures will be applied after ANOVA. The potential synergistic effect between treatments is analyzed by two-way ANOVA. All data are analyzed using SPSS 17.0, p<0 05 is considered to be statistically significant.

### Example 3

### In Vivo Anti-Tumor Efficacy of BT1718 in the Treatment of MT1-MMP Low-Expressing PDX Model in Female Balb/C Nude Mice

### Study Objective

The objective of this study is to evaluate the in vivo anti-tumor efficacy of BT1718 in the treatment of a MT1-MMP low-expressing cell line PDX model in female Balb/C nude mice

### Materials: Animal and Housing Conditions

### Animals

Species: Mus Musculus

Strain: Balb/C nude

Age: 6-8 weeks

Sex: female

Body weight: 18-22 g

Number of animals: 18 mice plus spare

### Housing Conditions

The mice are kept in individual ventilation cages at constant temperature and humidity with 3 animals in each cage.

Temperature: 20˜26° C.

Humidity 40-70%.

Cages: Made of polycarbonate. The size is 300 mm×180 mm×150 mm. The bedding material is corn cob, which is changed twice per week.

Diet: Animals have free access to irradiation sterilized dry granule food during the entire study period.

Water: Animals have free access to sterile drinking water.

Cage identification: The identification labels for each cage contain the following information: number of animals, sex, strain, the date received, treatment, study number, group number and the starting date of the treatment.

Animal identification: Animals are marked by ear coding.

### Experimental Methods and Procedures

### Tumor Inoculation

Each mouse is inoculated subcutaneously at the right flank with a tumor fragment (30 mm³) for tumor development. The treatments is started when the average tumor volume reaches 164 mm³.

### Observations

All the procedures related to animal handling, care and the treatment in the study are performed following the guidance of the Association for Assessment and Accreditation of Laboratory Animal Care (AAALAC). At the time of routine monitoring, the animals are daily checked for any effects of tumor growth and treatments on normal behavior such as mobility, food and water consumption (by looking only), body weight gain/loss (body weights are measured twice weekly), eye/hair matting and any other abnormal effect as stated in the protocol. Death and observed clinical signs are recorded on the basis of the numbers of animals within each subset.

### Tumor Measurements and the Endpoints

The major endpoint is to see if the tumor growth could be delayed or mice could be cured. Tumor size is measured two times weekly in two dimensions using a caliper, and the volume is expressed in mm³ using the formula: V=0.5 a×b² where a and b are the long and short diameters of the tumor, respectively. The tumor size is then used for calculations of T/C value. The T/C value (in percent) is an indication of antitumor effectiveness; T and C are the mean volumes of the treated and control groups, respectively, on a given day.

TGI is calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

### Sample Collection

Re-dose mice in group 2, 3 and collect plasma at 5 min, 15 min, 30 min, 60 min and 120 min on day 24.

Collect the tumor samples and fix in 10% formalin, then embed in paraffin and store at ambient temperature.

### Statistical Analysis

Summary statistics, including mean and the standard error of the mean (SEM), are provided for the tumor volume of each group at each time point.

Statistical analysis of difference in tumor volume among the groups is conducted on the data obtained at the best therapeutic time point after the final dose.

A one-way ANOVA is performed to compare tumor volume among groups, and when a significant F-statistics (a ratio of treatment variance to the error variance) is obtained, comparisons between groups are carried out with Games-Howell test. All data are analyzed using Prism. P<0.05 is considered to be statistically significant.

### Example 4

### In Vivo Efficacy Test of BT1718 in the Treatment of a Syngeneic Model in C57BL/6 Mice

### Study Objective

The objective of this study is to evaluate the in vivo anti-tumor efficacy of BT1718 in the treatment of the subcutaneous syngeneic model of bladder, endometrial, esophageal, glioblastoma, mesothelioma, multiple myeloma, ovarian, pancreatic, and/or prostate cancer in C57BL/6 mice.

### Study Design

The study design is shown in Table 5, below.

TABLE 5
Experimental design

| | | Dosage | | Dosing | | |
|---|---|---|---|---|---|---|
| Group | BDCs | (mg/kg) | n | volume(ml/kg) | route | Schedule |
| 1 | Vehicle | — | 6 | 10 | i.v. | biw*2 weeks |
| 2 | BT1718 | 1 | 6 | 10 | i.v. | biw*2 weeks |
| 3 | BT1718 | 3 | 6 | 10 | i.v. | biw*2 weeks |
| 4 | BT1718 | 10 | 6 | 10 | i.v. | biw*2 weeks |

### Animals

Species: Mus Musculus

Strain: C57BL/6 mice

Age: 6-10 weeks

Sex: Female

Body weight: 18-22 g

Number of animals: 24 plus spare

### Housing Condition

The animals are kept in ventilation cages at constant temperature and humidity with 3 animals in each cage.

Temperature: 2026° C.

Humidity: 40-70%.

Cages: Made of polycarbonate. The size is 300 mm×180 mm×150 mm. The bedding material is corn cob, which is changed twice per week.

Diet: Animals have free access to irradiation sterilized dry granule food during the entire study period.

Water: Animals have free access to sterile drinking water.

Cage identification: The identification labels for each cage contain the following information: number of animals, sex, strain, the date received, treatment, study number, group number and the starting date of the treatment.

Animal identification: Animals are marked by ear coding.

### Test and Positive Control Articles

Product identification: BT1718

Manufacturer: Bicycle Therapeutics

Lot number: N/A

Physical description: Lyophilized powder

Molecular weight: 3511.4

Package and storage condition: store at −80° C.

### Cell Culture

The bladder, endometrial, esophageal, glioblastoma, mesothelioma, multiple myeloma, ovarian, pancreatic, and/or prostate cancer tumor cells are maintained in vitro as a monolayer culture in a suitable medium at 37° C. in an atmosphere of 5% CO₂ in air. The tumor cells are routinely subcultured twice weekly by trypsin-EDTA treatment. The cells growing in an exponential growth phase are harvested and counted for tumor inoculation.

### Tumor Inoculation

Each mouse is inoculated subcutaneously at the right flank with bladder, endometrial, esophageal, glioblastoma, mesothelioma, multiple myeloma, ovarian, pancreatic, and/or prostate cancer tumor cells (1×10⁶) in 0.1 ml of PBS for tumor development. The animals are randomized and treatment is started when the average tumor volume reaches approximately 105 mm³ for the efficacy study. The test article administration and the animal numbers in each group are shown in the experimental design table (Table 5).

### Testing Article Formulation Preparation

The formulation of the testing article is shown in Table 6, below.

TABLE 6
Testing Article Formulation Preparation

| Test | Con. | | |
|---|---|---|---|
| article | (mg/ml) | Formulation | Buffer |
| Vehicle | — | 25 mM Histidine pH 7, 10% Sucrose | — |
| BT1718 | 1.0 | Add 10 mg BT1718 into 10 ml buffer, | 25 mM |
| | | sonicate and shake to ensure the | Histidine |
| | | solution to be clear | pH 7, 10% |
| | 0.3 | Add 240 ul 1.0 mg/ml BT1718 into | Sucrose |
| | | 560 ul buffer, shake to ensure | |
| | | the solution to be clear | |
| | 0.1 | Add 80 ul 1.0 mg/ml BT1718 into | |
| | | 720 ul buffer, shake to ensure the | |
| | | solution to be clear | |

### Observations

All the procedures related to animal handling, care and the treatment in the study are performed according to the guidelines of the Association for Assessment and Accreditation of Laboratory Animal Care (AAALAC). At the time of routine monitoring, the animals are daily checked for any effects of tumor growth and treatments on normal behavior such as mobility, food and water consumption (by looking only), body weight gain/loss (body weights are measured every day), eye/hair matting and any other abnormal effect as stated in the protocol. Death and observed clinical signs are recorded on the basis of the numbers of animals within each subset.

### Tumor Measurements and the Endpoints

The major endpoint is to see if the tumor growth could be delayed or mice could be cured. Tumor size is measured three times weekly in two dimensions using a caliper, and the volume is expressed in mm³ using the formula: V=0.5a×b2 where a and b are the long and short diameters of the tumor, respectively. The tumor size is then used for calculations of T/C value. The T/C value (in percent) is an indication of antitumor effectiveness; T and C are the mean volumes of the treated and control groups, respectively, on a given day.

TGI is calculated for each group using the formula: TGI (%)=[1−(Ti−T0)/(Vi−V0)]×00; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

### Statistical Analysis

Summary statistics, including mean and the standard error of the mean (SEM), are provided for the tumor volume of each group at each time point.

Statistical analysis of difference in tumor volume among the groups is conducted on the data obtained at the best therapeutic time point after the final dose.

A one-way ANOVA is performed to compare tumor volume among groups, and when a significant F-statistics (a ratio of treatment variance to the error variance) is obtained, comparisons between groups are carried out with Games-Howell test. All data are analyzed using Prism. P<0.05 is considered to be statistically significant.

### Example 5

### In Vivo Efficacy Test of BT1718 Alone or in Combination with Anti-PD-1 Antibody in the Treatment of a Syngeneic Model in C57BL/6 Mice

### Study Objective

The objective of the research is to evaluate the in vivo anti-tumor efficacy of BT1718 alone or in combination with Anti-PD-1 antibody in the treatment of the subcutaneous syngeneic model of bladder, endometrial, esophageal, glioblastoma, mesothelioma, multiple myeloma, ovarian, pancreatic, and/or prostate cancer in C57BL/6 mice.

### Experimental Design

TABLE 7
Experimental design

| Gr | n | I/O | Dose(mg/kg) | Frequency | compound | Dose(mg/kg) | frequency |
|---|---|---|---|---|---|---|---|
| 1 | 6 | Vehicle | — | biw*2 | Vehicle | — | biw*2 weeks |
| | | | | weeks | | | |
| 2 | 6 | Vehicle | — | biw*2 | BT1718 | 3 | biw*2 weeks |
| | | | | weeks | | | |
| 3 | 6 | Vehicle | — | biw*2 | BT1718 | 10 | biw*2 weeks |
| | | | | weeks | | | |
| 4 | 6 | aPD-1 | 10 | biw*2 | Vehicle | — | biw*2 weeks |
| | | | | weeks | | | |
| 5 | 6 | aPD-1 | 10 | biw*2 | BT1718 | 3 | biw*2 weeks |
| | | | | weeks | | | |
| 6 | 6 | aPD-1 | 10 | biw*2 | BT1718 | 10 | biw*2 weeks |
| | | | | weeks | | | |

### Materials

### Animals

Species: Mus Musculus

Strain: C57BL/6 mice

Age: 6-10 weeks

Sex: Female

Body weight: 18-22 g

Number of animals: 36 plus spare

### Housing Condition

The animals are kept in ventilation cages at constant temperature and humidity with 3 animals in each cage.

Temperature: 2026° C.

Humidity: 40-70%.

Cages: Made of polycarbonate. The size is 300 mm×180 mm×150 mm. The bedding material is corn cob, which is changed twice per week.

Diet: Animals have free access to irradiation sterilized dry granule food during the entire study period.

Water: Animals have free access to sterile drinking water.

Cage identification: The identification labels for each cage contain the following information: number of animals, sex, strain, the date received, treatment, study number, group number and the starting date of the treatment.

Animal identification: Animals are marked by ear coding.

### Test and Positive Control Articles

Product identification: BT1718

Manufacturer: Bicycle Therapeutics

Lot number: N/A

Physical description: Lyophilized powder

Molecular weight: 3511.4

Package and storage condition: store at −80° C.

Product identification: Anti-PD-1 antibody

Physical description: Liquid

Concentration: 11.5 mg/ml

Package and storage condition: store at −80° C.

### Experimental Methods and Procedures

### Cell Culture

The tumor cells are maintained in vitro as a monolayer culture in a suitable medium at 37° C. in an atmosphere of 5% CO₂ in air. The tumor cells are routinely subcultured twice weekly by trypsin-EDTA treatment. The cells growing in an exponential growth phase are harvested and counted for tumor inoculation.

### Tumor Inoculation

Each mouse is inoculated subcutaneously at the right flank with bladder, endometrial, esophageal, glioblastoma, mesothelioma, multiple myeloma, ovarian, pancreatic, or prostate cancer tumor cells (1×10⁶) in 0.1 ml of PBS for tumor development. The animals are randomized and treatment starts when the average tumor volume reaches approximately 105 mm³ for the efficacy study. The test article administration and the animal numbers in each group are shown in the experimental design table (Table 8).

TABLE 8
Testing Article Formulation Preparation

| Test | Con. | | |
|---|---|---|---|
| article | (mg/ml) | Formulation | Buffer |
| Vehicle1 | — | 25 mM Histidine pH 7, 10% Sucrose | — |
| Vehicle2 | — | 20 mM Histidine pH 5, 5% Sucrose | — |
| BT1718 | 1.0 | Add 10 mg BT1718 into 10 ml buffer, | 25 mM |
| | | sonicate and shake to ensure the | Histidine |
| | | solution to be clear | pH 7, 10% |
| | 0.3 | Add 900 ul 1.0 mg/ml BT1718 into | Sucrose |
| | | 2.1 ml buffer, shake to ensure | |
| | | the solution to be clear | |
| | 0.1 | Add 300 ul 1.0 mg/ml BT1718 into | |
| | | 2.7 ml buffer, shake to ensure | |
| | | the solution to be clear | |
| PD-1 | 1 | Add 348 ul 11.5 mg/ml PD-1 into | 20 mM |
| | | 3.652 ml buffer, shake to ensure | Histidine |
| | | the solution to be clear | pH 5, 5% |
| | | | Sucrose |

### Observations

All the procedures related to animal handling, care and the treatment in the study are performed according to the guidelines of the Association for Assessment and Accreditation of Laboratory Animal Care (AAALAC). At the time of routine monitoring, the animals are daily checked for any effects of tumor growth and treatments on normal behavior such as mobility, food and water consumption (by looking only), body weight gain/loss (body weights are measured every day), eye/hair matting and any other abnormal effect as stated in the protocol. Death and observed clinical signs are recorded on the basis of the numbers of animals within each subset.

### Tumor Measurements and the Endpoints

The major endpoint is to see if the tumor growth could be delayed or mice could be cured. Tumor size is measured three times weekly in two dimensions using a caliper, and the volume is expressed in mm³ using the formula: V=0.5a×b2 where a and b are the long and short diameters of the tumor, respectively. The tumor size is then used for calculations of T/C value. The T/C value (in percent) is an indication of antitumor effectiveness; T and C are the mean volumes of the treated and control groups, respectively, on a given day.

TGI is calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

### Statistical Analysis

Summary statistics, including mean and the standard error of the mean (SEM), are provided for the tumor volume of each group at each time point.

Statistical analysis of difference in tumor volume among the groups is conducted on the data obtained at the best therapeutic time point after the final dose.

A one-way ANOVA is performed to compare tumor volume among groups, and when a significant F-statistics (a ratio of treatment variance to the error variance) is obtained, comparisons between groups were carried out with Games-Howell test. All data are analyzed using Prism. P<0.05 is considered to be statistically significant.

### Example 6

### In Vivo Efficacy Test of BT1718 in Treatment of HT1080 Xenograft in Balb/c Nude Mice

### Study Objective

The objective of the research was to evaluate the in vivo anti-tumor efficacy of BT1718 in treatment of HT1080 xenograft model in Balb/c nude mice.

### Experimental Design

TABLE 9
Experimental Design

| | | Dosage | | Dosing Volume | Dosing | |
|---|---|---|---|---|---|---|
| Gr | Treatment | (mg/kg) | n | (ml/kg) | Route | Schedule |
| 1 | Vehicle | — | 5 | 10 | i.v. | biw*4 weeks |
| 2 | BT1718 | 1.6 | 5 | 10 | i.v. | biw*4 weeks |
| 3 | BT1718 | 2.4 | 5 | 10 | i.v. | qw*4 weeks |
| 4 | BT1718 | 2.4 | 5 | 10 | i.v. | biw*4 weeks |
| 5 | BT1718 | 3.2 | 5 | 10 | i.v. | qw*4 weeks |
| 6 | BT1718 | 4.8 | 5 | 10 | i.v. | qw*4 weeks |
| 7 | BT1718 | 6.4 | 5 | 10 | i.v. | qw*4 weeks |
| 8 | BT1718 | 9.6 | 5 | 10 | i.v. | qw*4 weeks |

### Materials

### Animals

Species: Mus Musculus; Strain: Balb/c nude; Age: 6-8 weeks; Sex: female Body weight: 18-22 g; Number of animals: 40 mice plus spare;

### Housing Condition

The mice were kept in individual ventilation cages at constant temperature and humidity with 5 animals in each cage.

Temperature: 20˜26° C.

Humidity 40-70%.

Cages: Made of polycarbonate. The size is 300 mm×180 mm×150 mm. The bedding material is corn cob, which is changed twice per week.

Diet: Animals had free access to irradiation sterilized dry granule food during the entire study period.

Water: Animals had free access to sterile drinking water.

Cage identification: The identification labels for each cage contained the following information: number of animals, sex, strain, the date received, treatment, study number, group number and the starting date of the treatment.

Animal identification: Animals were marked by ear coding.

### Test and Positive Control Articles

Product identification: BT1718; Manufacturer: Bicycle Therapeutics; Lot number: 02; Physical description: Lyophilised powder; Molecular weight: 3511.4; Purity: 97.70%; Package and storage condition: stored at −80° C.

### Experimental Methods and Procedures

### Cell Culture

The cells growing in an exponential growth phase were harvested and counted for tumor inoculation.

### Tumor Inoculation

Each mouse was inoculated subcutaneously at the right flank with HT1080 tumor cells (5×10\hat{ }6) in 0.2 ml of PBS for tumor development. 40 animals were randomized when the average tumor volume reached 155 mm³. The test article administration and the animal numbers in each group were shown in the experimental design table.

TABLE 10
Testing Article Formulation Preparation

| | Dose | |
|---|---|---|
| Treatment | (mg/ml) | Formulation |
| Vehicle | — | 25 mM Histidine, 10% sucrose |
| BT1718 | 0.96 | Dissolve 22.72 mg BT1718 in 23.122 ml |
| | | Histidine buffer. |
| | 0.16 | Dilute 1.733 ml 0.96 mg/ml BT1718 with |
| | | 8.672 ml buffer. |
| | 0.24 | Dilute 3.9 ml 0.96 mg/ml BT1718 with |
| | | 11.7 ml buffer. |
| | 0.32 | Dilute 1.732 ml 0.96 mg/ml BT1718 with |
| | | 3.468 ml buffer. |
| | 0.48 | Dilute 2.6 ml 0.96 mg/ml BT1718 with |
| | | 2.6 ml buffer. |
| | 0.64 | Dilute 3.468 ml 0.96 mg/ml BT1718 with |
| | | 1.732 ml buffer. |

### Observations

All the procedures related to animal handling, care and the treatment in the study were performed according to the guidelines of the Association for Assessment and Accreditation of Laboratory Animal Care (AAALAC). At the time of routine monitoring, the animals were checked for any effects of tumor growth and treatments on normal behavior such as mobility, food and water consumption (by looking only), body-weight gain/loss, eye/hair matting and any other abnormal effect as stated in the protocol. Death and observed clinical signs were recorded on the basis of the numbers of animals within each subset.

### Tumor Measurements and the Endpoints

The major endpoint was to see if the tumor growth could be delayed or mice could be cured. Tumor volume was measured 3 times per week in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1-(Tᵢ−T₀)/(Vᵢ−V₀)]×100; Tᵢ is the average tumor volume of a treatment group on a given day, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

### Sample Collection

On PG-D21, 30 ul plasma was collected at 20 min post dosing.

### Statistical Analysis

Summary statistics, including mean and the standard error of the mean (SEM), are provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, comparisons between groups were carried out with Games-Howell test. All data were analyzed using GraphPad Prism 5.0. P<0.05 was considered to be statistically significant.

### Results

Body Weight change and Tumor Growth Curve

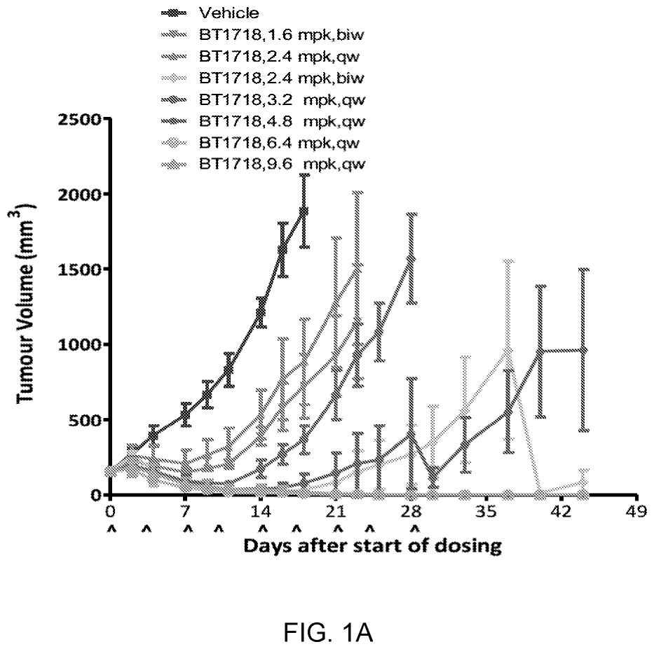

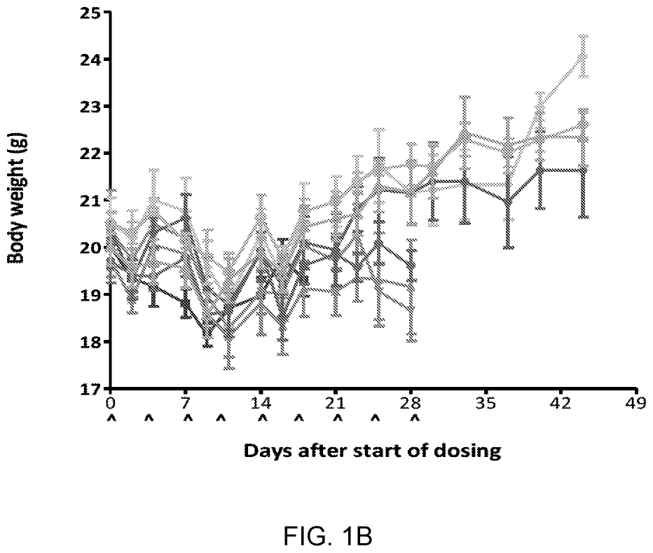

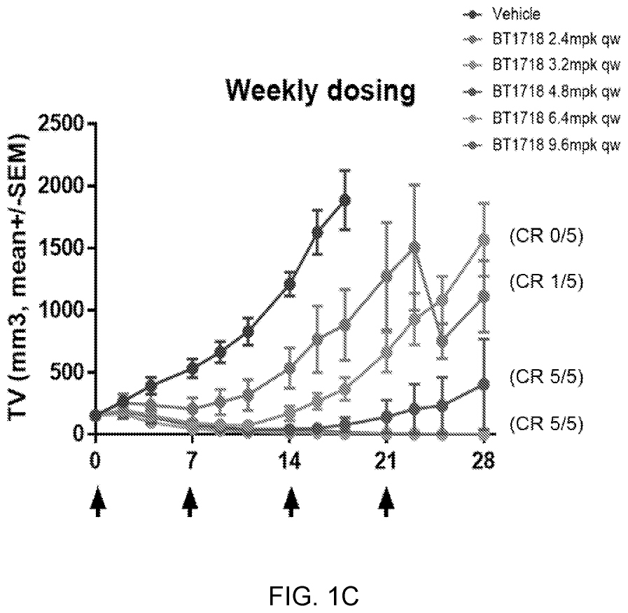

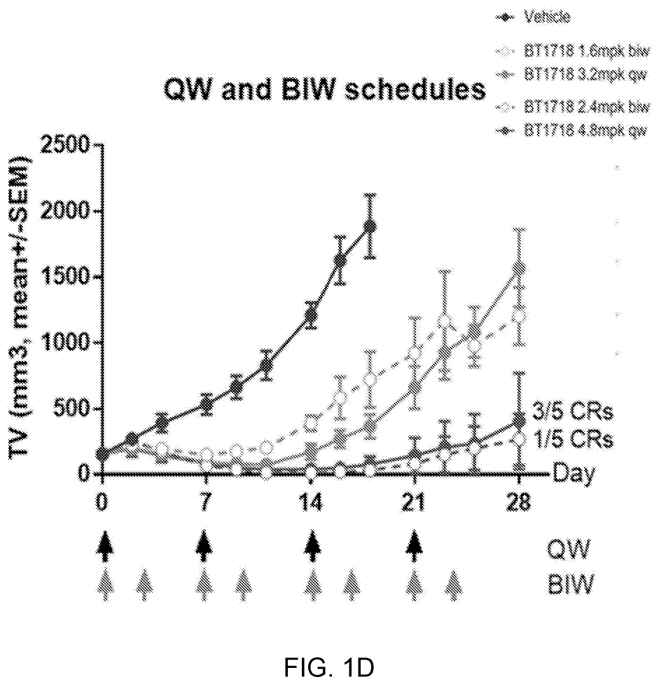

Body weight and tumor growth are shown in FIG. 1.

The data show that, in HT1080 model, BT1718 is efficacious when dosed once weekly (QW); Comparable efficacy observed with weekly and twice weekly dosing (BIW) at the same total dose; QW dosing efficacy maximized at 6.4 mg/kg; in clinic, weekly dosing at higher dose likely possible and desirable.

### Tumor Volume Trace

Mean tumor volume over time in female Balb/c nude mice bearing HT1080 xenograft is shown in Tables 11 and 12.

TABLE 11
Tumor volume trace over time

| | | Days after the start of treatment | | | | | | | | |
|---|---|---|---|---|---|---|---|---|---|---|
| Gr. | Treatment | 0 | 2 | 4 | 7 | 9 | 11 | 14 | 16 | 18 |
| 1 | Vehicle, biw | 155 ± 14 | 271 ± 38 | 393 ± 70 | 534 ± 76 | 666 ± 86 | 830 ± 109 | 121 ± 96 | 1628 ± 177 | 1886 ± 239 |
| 2 | BT1718, | 155 ± 19 | 226 ± 27 | 192 ± 30 | 149 ± 25 | 176 ± 18 | 203 ± 27 | 390 ± 58 | 584 ± 158 | 724 ± 211 |
| | 1.6 mpk, biw | | | | | | | | | |
| 3 | BT1718, | 155 ± 21 | 260 ± 69 | 238 ± 90 | 206 ± 91 | 264 ± 104 | 321 ± 123 | 536 ± 163 | 768 ± 268 | 885 ± 284 |
| | 2.4 mpk, qw | | | | | | | | | |
| 4 | BT1718, | 155 ± 18 | 259 ± 31 | 173 ± 28 | 68 ± 13 | 39 ± 8 | 17 ± 4 | 13 ± 8 | 22 ± 18 | 32 ± 30 |
| | 2.4 mpk, biw | | | | | | | | | |
| 5 | BT1718, | 155 ± 19 | 199 ± 34 | 156 ± 33 | 92 ± 8 | 80 ± 17 | 73 ± 22 | 173 ± 59 | 269 ± 65 | 368 ± 93 |
| | 3.2 mpk, qw | | | | | | | | | |
| 6 | BT1718, | 155 ± 21 | 194 ± 58 | 146 ± 53 | 88 ± 46 | 54 ± 30 | 39 ± 22 | 40 ± 25 | 48 ± 31 | 78 ± 61 |
| | 4.8 mpk, qw | | | | | | | | | |
| 7 | BT1718, | 155 ± 23 | 169 ± 47 | 98 ± 38 | 46 ± 20 | 32 ± 21 | 20 ± 14 | 15 ± 12 | 13 ± 11 | 7 ± 7 |
| | 6.4 mpk, qw | | | | | | | | | |
| 8 | BT1718, | 155 ± 27 | 186 ± 55 | 144 ± 54 | 72 ± 41 | 58 ± 39 | 38 ± 27 | 36 ± 27 | 31 ± 25 | 15 ± 15 |
| | 9.6 mpk, qw | | | | | | | | | |

TABLE 12
Tumor volume trace over time

| | | Days after the start of treatment | | | | | | | | |
|---|---|---|---|---|---|---|---|---|---|---|
| Gr. | Treatment | 21 | 23 | 25 | 28 | 30 | 33 | 37 | 40 | 44 |
| 1 | Vehicle, biw | — | — | — | — | — | — | — | — | — |
| 2 | BT1718, | 924 ± 288 | 1167 ± 377 | 981 ± 157 | 1205 ± 217 | — | — | — | — | — |
| | 1.6 mpk, biw | | | | | | | | | |
| 3 | BT1718, | 1277 ± 432 | 1505 ± 504 | 755 ± 142 | 1114 ± 287 | — | — | — | — | — |
| | 2.4 mpk, qw | | | | | | | | | |
| 4 | BT1718, | 83 ± 77 | 152 ± 139 | 199 ± 164 | 267 ± 196 | 348 ± 242 | 564 ± 350 | 960 ± 591 | 12 ± 12 | 82 ± 82 |
| | 2.4 mpk, biw | | | | | | | | | |
| 5 | BT1718, | 664 ± 161 | 930 ± 207 | 1081 ± 191 | 1568 ± 295 | — | — | — | — | — |
| | 3.2 mpk, qw | | | | | | | | | |
| 6 | BT1718, | 145 ± 134 | 208 ± 201 | 234 ± 228 | 405 ± 366 | 115 ± 65 | 333 ± 182 | 553 ± 272 | 952 ± 434 | 96 ± 1534 |
| | 4.8 mpk, qw | | | | | | | | | |
| 7 | BT1718, | 4 ± 4 | 4 ± 4 | 2 ± 2 | 0 ± 0 | 0 ± 0 | 0 ± 0 | 0 ± 0 | 0 ± 0 | 2 ± 2 |
| | 6.4 mpk, qw | | | | | | | | | |
| 8 | BT1718, | 8 ± 8 | 7 ± 7 | 0 ± 0 | 0 ± 0 | 0 ± 0 | 0 ± 0 | 0 ± 0 | 0 ± 0 | 0 ± 0 |
| | 9.6 mpk, qw | | | | | | | | | |

### Tumor Growth Inhibition Analysis

Tumor growth inhibition rate of BT1718 in the HT1080 xenograft model was calculated based on tumor volume measurements on day 18 after the start of treatment.

TABLE 13
Tumor growth inhibition analysis

| | | Tumor | | | |
|---|---|---|---|---|---|
| | | Volume | T/C^{b} | TGI | |
| Gr | Treatment | (mm³)^{a} | (%) | (%) | P value |
| 1 | Vehicle, biw | 1886 ± 239 | — | — | — |
| 2 | BT1718, 1.6 mpk, biw | 724 ± 211 | 38.4 | 67.1 | p < 0.001 |
| 3 | BT1718, 2.4 mpk, qw | 885 ± 284 | 46.9 | 57.8 | p < 0.001 |
| 4 | BT1718, 2.4 mpk, biw | 32 ± 30 | 1.7 | 107.1 | p < 0.001 |
| 5 | BT1718, 3.2 mpk, qw | 368 ± 93 | 19.5 | 87.7 | p < 0.001 |
| 6 | BT1718, 4.8 mpk, qw | 78 ± 61 | 4.1 | 104.5 | p < 0.001 |
| 7 | BT1718, 6.4 mpk, qw | 7 ± 7 | 0.4 | 108.5 | p < 0.001 |
| 8 | BT1718, 9.6 mpk, qw | 15 ± 15 | 0.8 | 108.1 | p < 0.001 |

^{a}Mean ± SEM.
^{b}Tumor Growth Inhibition is calculated by dividing the group average tumor volume for the treated group by the group average tumor volume for the control group (T/C).

Results Summary and Discussion

In this study, the therapeutic efficacy of BT1718 in the HT1080 xenograft model was evaluated. The measured body weights and tumor volumes of all treatment groups at various time points are shown in FIG. 1 and Tables 11-13.

The mean tumor size of vehicle treated mice reached 1886 mm³ on day 18. BT1718 at 1.6 mg/kg biw (TV=724 mm³, TGI=67.1%, p<0.001), 2.4 mg/kg qw (TV=885 mm³, TGI=57.8%, p<0.001), 2.4 mg/kg biw (TV=32 mm³, TGI=107.1%, p<0.001), 3.2 mg/kg qw (TV=368 mm³, TGI=87.7%, p<0.001), 4.8 mg/kg qw (TV=78 mm³, TGI=104.5%, p<0.001), 6.4 mg/kg qw (TV=7 mm³, TGI=108.5%, p<0.001) and 9.6 mg/kg qw (TV=15 mm³, TGI=108.1%, p<0.001) produced dose dependent or dose frequency dependent antitumor activity on day 18. Among them, BT1718 at 6.4 mg/kg qw and BT1718 at 9.6 mg/kg qw completely regressed the tumors on day 30, and one tumor in BT1718 6.4 mg/kg qw group showed relapse on day 44.

In this study, some mice lost over 10% bodyweight loss under the burden of HT1080 tumor.

### Example 7

### MMP14 Immunohistochemistry (MC) Analysis of Tumor Microarrays (TMA) Using a Novel Scoring System Guides Selection of Patient Populations for BT1718 Expansion Cohorts

Introduction: BT1718 is a targeted Bicycle peptide-conjugate designed to deliver the anti-tubulin agent, DM1 to tumors expressing membrane type 1-matrix metalloprotease (MT1-MMP; MMP14; MT1). In vivo preclinical BT1718 efficacy data demonstrated that anti-tumor activity of BT1718 is dependent on the level of tumor MMP14 expression. In patient tumors MMP14 expression has been reported in tumor and/or stromal cells, both of which may contribute to the potential for anti-tumor effects following BT1718 dosing. BT1718 is currently being investigated in a Phase ½ clinical trial, which includes both dose escalation (ongoing) and dose expansion cohorts enrolling patients with advanced solid tumors that have exhausted standard therapeutic options. The dose expansion cohorts will enroll patients with tumors expressing high levels of MMP14 following establishment of the recommended Phase 2 dose. Here we describe the analysis of TMA's stained with a clinical grade MMP14 IHC assay to guide which patient populations to include in BT1718 dose expansion cohorts.

Methods: A clinical grade MMP14 IHC assay was developed on the Ventana platform using MMP14 primary antibody (MAB3328, Millipore) at 1:6000, with Optiview detection chemistry. Cancer indications reported in the literature with high MMP14 expression including ovarian, bladder, triple negative breast, esophageal, and NSCLC were stained and MMP14 expression levels estimated by consensus review of two pathologists using an H-score scale (staining intensity*percent positivity). H-scores (0-300) were derived separately for tumor membrane (TM), cytoplasm (TC), and stroma (TS) for each case.

Additionally, SCLC were stained and MMP14 expression levels estimated by consensus review of two pathologists as described above.

Results: TM/TS, but not TC, H-scores were analyzed to identify BT1718 dose expansion cohorts as MMP14 expression in these locations is likely to have the greatest potential for BT1718 binding leading to anti-tumor activity. Histogram analyses were generated separately for TM and TS with a bin-width of 50. The distribution of MMP14 staining in TM and TS was different (Table 14) with H-score between 0-49 being the most frequently populated TM bin, regardless of indication. In contrast, TS scores were typically higher (e.g. H-score=100-149), with the clearest example being ovarian cancer (89% of cases TM 0-49 & only 20% of cases TS 0-49). In addition, within lung, tumor subtype analysis demonstrated that cases of squamous histology appeared enriched for higher TM H-scores (TM≥150=36%) compared to adenocarcinoma cases (TM≥150=2%). Moreover, various TM and/or TS H-score boundaries were modelled with the aim of delivering a proposed cut-off for recruiting patients with high MMP14 expression to the expansion cohorts, the results of this modelling will be presented.

Specifically, within NSCLC, tumor subtype analysis demonstrated that cases of squamous histology appeared enriched for higher TM H-scores (TM≥150=36%) compared to adenocarcinoma cases (TM≥150=2%).

TABLE 14
Distribution of MMP14 Tumor Membrane and Tumor H-score distribution across multiple indications

| | Count | Tumor Membrane (%) | | | | | | Tumor Stromal (%) | | | | | |
|---|---|---|---|---|---|---|---|---|---|---|---|---|---|
| | N | 0-49 | 50-99 | 100-149 | 150-199 | 200-249 | 250+ | 0-49 | 50-99 | 100-149 | 150-199 | 200-249 | 250+ |
| NSCLC_SQUAM | 75 | 40 | 11 | 13 | 11 | 13 | 12 | 8 | 4 | 32 | 19 | 15 | 23 |
| NSCLC_ADENO | 69 | 91 | 4 | 1 | 0 | 1 | 1 | 13 | 13 | 36 | 12 | 14 | 12 |
| ESOPHAGEAL_SQUAM | 66 | 45 | 14 | 23 | 14 | 0 | 5 | 5 | 11 | 21 | 20 | 20 | 24 |
| OVARIAN | 82 | 89 | 1 | 6 | 1 | 1 | 1 | 20 | 17 | 35 | 7 | 16 | 5 |
| TNBC | 81 | 57 | 11 | 11 | 4 | 9 | 9 | 15 | 23 | 19 | 17 | 12 | 14 |
| BLADDER UROTHELIAL | 96 | 44 | 17 | 19 | 11 | 6 | 3 | 18 | 19 | 34 | 15 | 8 | 6 |

Conclusion: Based on these analyses; squamous lung, squamous esophageal and an all comers high MMP14 basket were selected (using a TM cut-off of at least H-score ≥100) as the first set of BT1718 expansion cohorts.

Additional MMP14 Immunohistochemistry (IHC) analysis of tumor microarrays (TMA) was conducted on two SCLC TMAs. The results from these SCLC TMAs as well as the results from the NSCLC TMAs shown above (Table 14) are depicted in FIG. 2 and Tables 15 and 16, below. Note that an additional lung squamous case was identified resulting in the count for NSCLC squam being 76 rather than 75 as shown in Table 14.

TABLE 15
Distribution of MMP14 Tumor Membrane and Tumor H-
score distribution across multiple indications.

| | Count | Tumor Membrane (%) | | | | | |
|---|---|---|---|---|---|---|---|
| | N | 0-49 | 50-99 | 100-149 | 150-199 | 200-249 | 250+ |
| NSCLC_squam | 76 | 41 | 11 | 12 | 12 | 13 | 12 |
| NSCLC_adeno | 69 | 91 | 4 | 1 | 0 | 1 | 1 |
| SCLC | 75 | 81 | 5 | 4 | 8 | 1 | 0 |
| Esophageal_squam | 66 | 45 | 14 | 23 | 14 | 0 | 5 |
| Ovarian | 82 | 89 | 1 | 6 | 1 | 1 | 1 |
| TNBC | 81 | 57 | 11 | 11 | 4 | 9 | 9 |
| Bladder urothelial | 96 | 44 | 17 | 19 | 11 | 6 | 3 |

TABLE 16
Distribution of MMP14 Tumor Stromal and Tumor H-
score distribution across multiple indications.

| | Count | Tumor Stromal (%) | | | | | |
|---|---|---|---|---|---|---|---|
| | N | 0-49 | 50-99 | 100-149 | 150-199 | 200-249 | 250+ |
| NSCLC_squam | 76 | 8 | 4 | 32 | 20 | 14 | 22 |
| NSCLC_adeno | 69 | 13 | 13 | 36 | 12 | 14 | 12 |
| SCLC | 75 | 5 | 25 | 27 | 12 | 12 | 19 |
| Esophageal_squam | 66 | 5 | 11 | 21 | 20 | 20 | 24 |
| Ovarian | 82 | 20 | 17 | 35 | 7 | 16 | 5 |
| TNBC | 81 | 15 | 23 | 19 | 17 | 12 | 14 |
| Bladder urothelial | 96 | 18 | 19 | 34 | 15 | 8 | 6 |

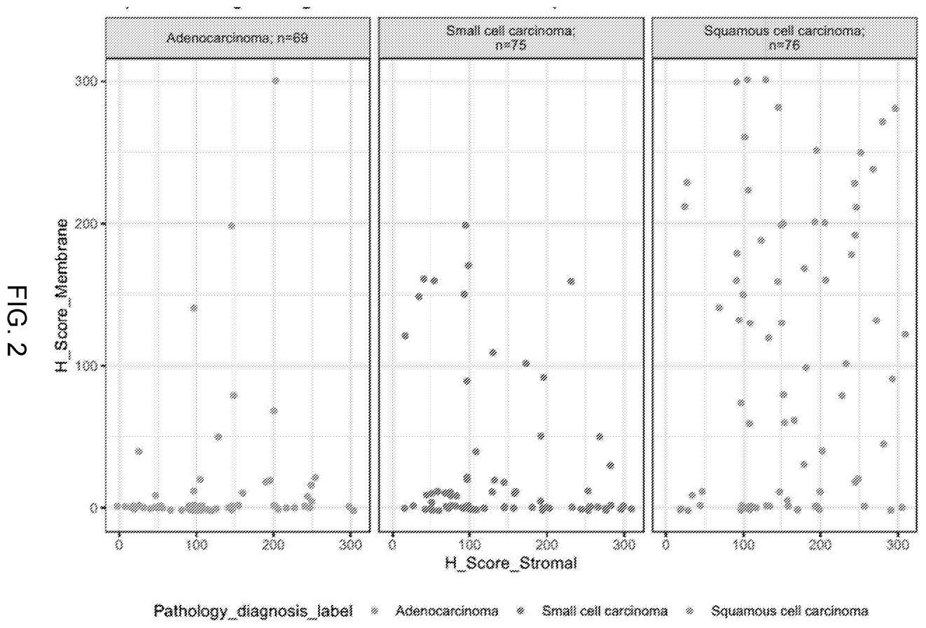

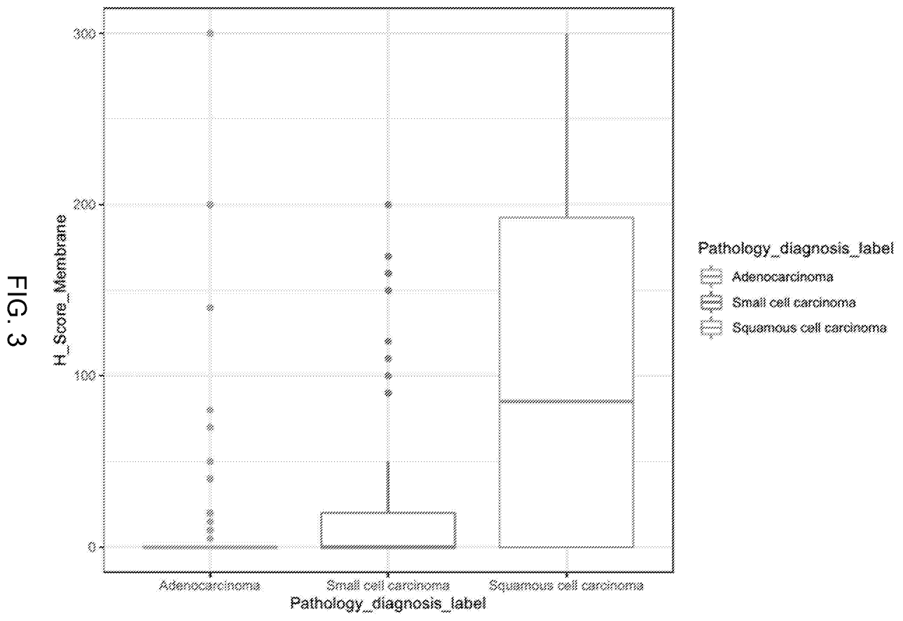

FIG. 3 depicts a box plot analysis of the tumor membrane (TM) H-scores presented in Table 15. Table 17 below presents the analysis presented in FIG. 3 in tabular form.

TABLE 17
Distribution of MMP14 Tumor Membrane and Tumor H-
score distribution across multiple indications.

| Pathology diagnosis | | | 25th | | | 75th | |
|---|---|---|---|---|---|---|---|
| label | count | min | percentile | mean | median | percentile | max |
| Adenocarcinoma | 69 | 0 | 0 | 15 | 0 | 0.0 | 300 |
| Small cell carcinoma | 75 | 0 | 0 | 27 | 0 | 20.0 | 200 |
| Squamous cell carcinoma | 76 | 0 | 0 | 106 | 85 | 192.5 | 300 |

MT1-MMP Immunohistochemistry Assay Protocol

### Equipment and Materials

### Equipment

Staining was performed on the Ventana Benchmark Ultra automated IHC/ISH instruments.

### Primary Antibody

The primary antibody to be used is:

Supplier: Merck Millipore

Clone: LEM-2/15.8

Catalogue no: MAB3328

Lot no: 3074991

Concentration: 1 mg/ml

Storage: 2-8° C.

### Additional Reagents

Additional reagents are listed below:
Dewaxing reagent: EZ Prep (Ventana, Ref 950-102)
Cell conditioning Solutions: Ultra CC1 (high pH—Ventana, Ref. 950-224)
Endogenous peroxidase inhibitor: OptiView DAB IHC Detection Kit (Ventana, Ref 760-700)
Antibody diluent: Menarini Universal RTU Antibody Diluent (MenaPath, Ref. MP-900-100)
IHC Detection/chromogen: OptiView DAB IHC Detection Kit (Ventana, Ref. 760-700)
Counterstain: Hematoxylin II (Ventana, Ref. 790-2208) and Bluing Reagent (Ventana, Ref 760-2037)

### Quantitation of Staining

Tumour cell membrane expression of MT1-MMP is quantified using a standard H score:

H=(1×% cells with 1+staining)+(2×% cells with 2+staining)+(3×% cells with 3+staining) Range=0-300.

Stromal expression of MT1-MMP is quantified using a standard H score:

H=(1×% cells with 1+staining)+(2×% cells with 2+staining)+(3×% cells with 3+staining) Range=0-300.

The MT1-MMP immunohistochemistry assay protocol is shown in Table 18 below.

TABLE 18
MT1-MMP immunohistochemistry assay protocol.

| Step | |
|---|---|
| Number | Procedure Step |
| 1 | Enable Mixers |
| 2 | Disable Mixers |
| 3 | [72° C. is the standard temperature] |
| 4 | Warmup Slide to [72 Deg C.] from Medium |
| | Temperatures (Deparaffinization) |
| 5 | Incubate for 4 Minutes |
| 6 | Apply EZPrep Volume Adjust |
| 7 | Rinse Slide With EZ Prep |
| 8 | Apply EZPrep Volume Adjust |
| 9 | Apply Coverslip |
| 10 | Rinse Slide With EZ Prep |
| 11 | Apply EZPrep Volume Adjust |
| 12 | Apply Coverslip |
| 13 | Rinse Slide With EZ Prep |
| 14 | Apply EZPrep Volume Adjust |
| 15 | Apply Coverslip |
| 16 | Rinse Slide With EZ Prep |
| 17 | Apply Depar Volume Adjust |
| 18 | Apply Coverslip |
| 19 | Enable Mixers |
| 20 | Disable Slide Heater |
| 21 | Pause Point (Landing Zone) |
| 22 | Rinse Slide With EZ Prep |
| 23 | Apply Long Cell Conditioner #1 |
| 24 | Apply CC Coverslip Long |
| 25 | [100° C. is the standard temperature] |
| 26 | Warmup Slide to [100 Deg C.], and Incubate |
| | for 4 Minutes (Cell Conditioner #1) |
| 27 | Incubate for 4 Minutes |
| 28 | Incubate for 8 Minutes |
| 29 | Apply Cell Conditioner #1 |
| 30 | Apply CC Medium Coverslip No BB |
| 31 | Incubate for 8 Minutes |
| 32 | Apply Cell Conditioner #1 |
| 33 | Apply CC Medium Coverslip No BB |
| 34 | Apply Cell Conditioner #1 |
| 35 | Apply CC Medium Coverslip No BB |
| 36 | Apply Cell Conditioner #1 |
| 37 | Apply CC Medium Coverslip No BB |
| 38 | Disable Slide Heater |
| 39 | Apply Cell Conditioner #1 |
| 40 | Apply CC Medium Coverslip No BB |
| 41 | Rinse Slide With Reaction Buffer |
| 42 | Adjust Slide Volume With Reaction Buffer |
| 43 | Apply Coverslip |
| 44 | Pause Point (Landing Zone) |
| 45 | Warmup Slide to 36 Deg C. |
| 46 | Rinse Slide With Reaction Buffer |
| 47 | Adjust Slide Volume With Reaction Buffer |
| 48 | Apply One Drop of OV PEROX IHBTR, Apply |
| | Coverslip, and Incubate for 4 Minutes |
| 49 | Rinse Slide With Reaction Buffer |
| 50 | Adjust Slide Volume With Reaction Buffer |
| 51 | Apply Coverslip |
| 52 | [If not selected, temperature will default to 36° C.] |
| 53 | Warmup Slide to 36 Deg C. |
| 54 | Rinse Slide With Reaction Buffer |
| 55 | Adjust Slide Volume With Reaction Buffer With Label Blow |
| | Off |
| 56 | Apply Coverslip |
| 57 | Hand Apply (Primary Antibody), and Incubate for [0 Hr 32 Min] |
| 58 | Warmup Slide to 36 Deg C. |
| 59 | Rinse Slide With Reaction Buffer |
| 60 | Apply 230 ul + VA Reaction Buffer |
| 61 | Apply Coverslip |
| 62 | Rinse Slide With Reaction Buffer |
| 63 | Apply 230 ul + VA Reaction Buffer |
| 64 | Apply Coverslip |
| 65 | Rinse Slide With Reaction Buffer |
| 66 | [If not selected, HQ Universal Linker will dispense |
| | and incubate 8 min in normal slide volume.] |
| 67 | Adjust Slide Volume With Reaction Buffer With Label Blow |
| | Off |
| 68 | Apply Coverslip, One Drop of OV HQ UNIV LINKR, |
| | and Incubate for 8 Minutes |
| 69 | Rinse Slide With Reaction Buffer |
| 70 | Apply 230 ul + VA Reaction Buffer |
| 71 | Apply Coverslip |
| 72 | Rinse Slide With Reaction Buffer |
| 73 | Apply 230 ul + VA Reaction Buffer |
| 74 | Apply Coverslip |
| 75 | Rinse Slide With Reaction Buffer |
| 76 | [If not selected, HRP Multimer will dispense |
| | and incubate 8 min in normal slide volume.] |
| 77 | Adjust Slide Volume With Reaction Buffer With Label Blow |
| | Off |
| 78 | Apply Coverslip, One Drop of OV HRP MULTIMER, |
| | and Incubate for 8 Minutes |
| 79 | Rinse Slide With Reaction Buffer |
| 80 | Apply 230 ul + VA Reaction Buffer |
| 81 | Apply Coverslip |
| 82 | Rinse Slide With Reaction Buffer |
| 83 | Apply 230 ul + VA Reaction Buffer |
| 84 | Apply Coverslip |
| 85 | Rinse Slide With Reaction Buffer |
| 86 | Adjust Slide Volume With Reaction Buffer |
| 87 | Apply One Drop of OV H2O2 and One Drop of OV DAB, |
| | Apply Coverslip, Incubate for 8 Minutes |
| 88 | Rinse Slide With Reaction Buffer |
| 89 | Adjust Slide Volume With Reaction Buffer |
| 90 | Apply One Drop of OV COPPER, Apply Coverslip, |
| | and Incubate for 4 Minutes |
| 91 | Rinse Slide With Reaction Buffer |
| 92 | Adjust Slide Volume With Reaction Buffer |
| 93 | Apply One Drop of [HEMATOXYLIN II] (Counterstain), |
| | Apply Coverslip, and Incubate for [8 Minutes] |
| 94 | Rinse Slide With Reaction Buffer |
| 95 | Adjust Slide Volume With Reaction Buffer |
| 96 | Apply Coverslip |
| 97 | Rinse Slide With Reaction Buffer |
| 98 | Adjust Slide Volume With Reaction Buffer |
| 99 | Apply One Drop of [BLUING REAGENT] (Post Counterstain), |
| | Apply Coverslip, and Incubate for [4 Minutes] |
| 100 | Rinse Slide With Reaction Buffer |
| 101 | Adjust Slide Volume With Reaction Buffer |
| 102 | Apply Coverslip |
| 103 | Disable Slide Heater |
| 104 | Rinse Slide With Reaction Buffer |

* One drop is one reagent dispense.

We claim:

1. A method of treating cancer in a patient, comprising administering to said patient a therapeutically effective amount of BT1718, or a pharmaceutically acceptable salt and/or composition thereof.

2. The method of claim 1, wherein the cancer is selected from the group consisting of bladder cancer, endometrial cancer, esophageal cancer, glioblastoma, mesothelioma, multiple myeloma, ovarian cancer, pancreatic cancer, lung cancer, and prostate cancer.

3. The method of claim 2, wherein the bladder cancer is selected from the group consisting of basal, p53-like, and luminal.

4. The method of claim 2, wherein the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated.

5. The method of claim 2, wherein the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI).

6. The method of claim 2, wherein the glioblastoma is selected from the group consisting of proneural, neural, classical, and mesenchymal.

7. The method of claim 2, wherein the mesothelioma is selected from the group consisting of pleural, peritoneal, pericardial, epithelioid, sarcomatoid, biphasic, and malignant.

8. The method of claim 2, wherein the multiple myeloma is selected from the group consisting of hyperdiploid, non-hyperdiploid, cyclin D translocation, MMSET translocation, MAF translocation, and unclassified.

9. The method of claim 2, wherein the ovarian cancer is selected from the group consisting of clear cell, endometrioid, mucinous, high-grade serous and low-grade serous.

10. The method of claim 2, wherein the pancreatic cancer is selected from the group consisting of squamous, pancreatic progenitor, immunogenic, and ADEX (Aberrantly Differentiated Endocrine eXocrine).

11. The method of claim 2, wherein the lung cancer is selected from the group consisting of non-small cell lung cancer (NSCLC) and small cell lung cancer (SCLC).

12. The method of claim 2, wherein the prostate cancer is selected from the group consisting of AZGP1 (subtype I), MUC1 (subtype II), and MUC1 (subtype III).

13. The method of claim 2, wherein the bladder cancer is high MT1-MMP expressing.

14. The method of claim 2, wherein the endometrial cancer is high MT1-MMP expressing.

15. The method of claim 2, wherein the esophageal cancer is high MT1-MMP expressing.

16. The method of claim 2, wherein the glioblastoma is high MT1-MMP expressing.

17. The method of claim 2, wherein the mesothelioma is high MT1-MMP expressing.

18. The method of claim 2, wherein the multiple myeloma is high MT1-MMP expressing.

19. The method of claim 2, wherein the ovarian cancer is high MT1-MMP expressing.

20. The method of claim 2, wherein the pancreatic cancer is high MT1-MMP expressing.

21. The method of claim 2, wherein the lung cancer is high MT1-MMP expressing.

22. The method of claim 2, wherein the prostate cancer is high MT1-MMP expressing.

23. A method of treating cancer selected from the group consisting of bladder cancer, endometrial cancer, esophageal cancer, glioblastoma, mesothelioma, multiple myeloma, ovarian cancer, pancreatic cancer, lung cancer, and prostate cancer associated with MT1-MMP in a patient, comprising administering to said patient a therapeutically effective amount of BT1718, or a pharmaceutically acceptable salt and/or composition thereof.

24. A method of treating cancer selected from the group consisting of bladder cancer, endometrial cancer, esophageal cancer, glioblastoma, mesothelioma, multiple myeloma, ovarian cancer, pancreatic cancer, lung cancer, and prostate cancer in a patient, comprising administering to said patient a minimally effective dose of BT1718, or a pharmaceutically acceptable salt and/or composition thereof, that is <3, 3, <10, or 10 mg/kg.

25. The method of claim 24, wherein the minimally effective dose is <10 mg/kg.

26. The method of claim 24, wherein the minimally effective dose is 3 mg/kg.

27. The method of claim 24, wherein the minimally effective dose is <3 mg/kg.

28. A method of identifying or selecting a patient having an elevated MT1-MMP level in a tumor tissue, comprising measuring MT1-MMP level in a tumor tissue of a patient, and selecting a patient having an elevated MT1-MMP level in the tumor tissue.

29. The method of claim 28, wherein the step of measuring MT1-MMP level in a tumor tissue of a patient comprises using an MT1-MMP immunohistochemistry (IHC) staining assay.

30. A method of identifying or selecting a patient having an elevated MT1-MMP level in a tumor tissue, comprising measuring staining intensity in a tumor tissue section of a patient using an MT1-MMP IHC staining assay, and selecting a patient who is staining positive in the MT1-MMP IHC staining assay.

31. The method of any one of claims 28-30, wherein the patient has bladder cancer, endometrial cancer, esophageal cancer, glioblastoma, mesothelioma, multiple myeloma, ovarian cancer, pancreatic cancer, lung cancer, or prostate cancer.

32. The method of claim 31, wherein the bladder cancer is selected from the group consisting of basal, p53-like, and luminal.

33. The method of claim 31, wherein the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated.

34. The method of claim 31, wherein the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI).

35. The method of claim 31, wherein the glioblastoma is selected from the group consisting of proneural, neural, classical, and mesenchymal.

36. The method of claim 31, wherein the mesothelioma is selected from the group consisting of pleural, peritoneal, pericardial, epithelioid, sarcomatoid, biphasic, and malignant.

37. The method of claim 31, wherein the multiple myeloma is selected from the group consisting of hyperdiploid, non-hyperdiploid, cyclin D translocation, MMSET translocation, MAF translocation, and unclassified.

38. The method of claim 31, wherein the ovarian cancer is selected from the group consisting of clear cell, endometrioid, mucinous, high-grade serous and low-grade serous.

39. The method of claim 31, wherein the pancreatic cancer is selected from the group consisting of squamous, pancreatic progenitor, immunogenic, and ADEX (Aberrantly Differentiated Endocrine eXocrine).

40. The method of claim 31, wherein the lung cancer is selected from the group consisting of non-small cell lung cancer (NSCLC) and small cell lung cancer (SCLC).

41. The method of claim 31, wherein the prostate cancer is selected from the group consisting of AZGP1 (subtype I), MUC1 (subtype II), and MUC1 (subtype III).

42. The method of claim 31, wherein the bladder cancer is high MT1-MMP expressing.

43. The method of claim 31, wherein the endometrial cancer is high MT1-MMP expressing.

44. The method of claim 31, wherein the esophageal cancer is high MT1-MMP expressing.

45. The method of claim 31, wherein the glioblastoma is high MT1-MMP expressing.

46. The method of claim 31, wherein the mesothelioma is high MT1-MMP expressing.

47. The method of claim 31, wherein the multiple myeloma is high MT1-MMP expressing.

48. The method of claim 31, wherein the ovarian cancer is high MT1-MMP expressing.

49. The method of claim 31, wherein the pancreatic cancer is high MT1-MMP expressing.

50. The method of claim 31, wherein the lung cancer is high MT1-MMP expressing.

51. The method of claim 31, wherein the prostate cancer is high MT1-MMP expressing.

52. The method of claim 29 or 30, wherein the MT1-MMP immunohistochemistry (IHC) staining assay uses a primary MT1-MMP antibody binding to the tumor membrane (TM) or tumor stroma (TS).

53. The method of claim 52, wherein the primary MT1-MMP antibody binding to the tumor membrane (TM) is primary MT1-MMP antibody MAB3328.

54. The method of claim 30, wherein staining positive in the MT1-MMP IHC staining assay refers to an H-score of about 15 or more, about 20 or more, about 30 or more, about 40 or more, about 50 or more, about 75 or more, about 100 or more, about 125 or more, about 150 or more, about 200 or more, or about 250 or more in a tumor tissue section in the MT1-MMP IHC staining assay.

55. The method of claim 30, wherein staining positive in the MT1-MMP IHC staining assay refers to an H-score for tumor cell membrane of about 15 or more, about 20 or more, about 30 or more, about 40 or more, about 50 or more, about 75 or more, about 100 or more, about 125 or more, about 150 or more, about 200 or more, or about 250 or more in a tumor tissue section in the MT1-MMP IHC staining assay.

56. The method of claim 30, wherein staining positive in the MT1-MMP IHC staining assay refers to an H-score for tumor stroma (TS) of about 15 or more, about 20 or more, about 30 or more, about 40 or more, about 50 or more, about 75 or more, about 100 or more, about 125 or more, about 150 or more, about 200 or more, or about 250 or more in a tumor tissue section in the MT1-MMP IHC staining assay.

57. A method of treating a cancer in a patient having an elevated MT1-MMP level in a tumor tissue, comprising administering to the patient a therapeutically effective amount of BT1718, or a pharmaceutically acceptable salt thereof, or a pharmaceutical composition thereof.

58. The method of claim 57, wherein a patient having an elevated MT1-MMP level in a tumor tissue refers to a patient having an H-score of about 15 or more, about 20 or more, about 30 or more, about 40 or more, about 50 or more, about 75 or more, about 100 or more, about 125 or more, about 150 or more, about 200 or more, or about 250 or more in a tumor tissue section in an MT1-MMP IHC staining assay.

59. A method of treating a cancer in a patient, comprising selecting a patient having an H-score of about 15 or more, about 20 or more, about 30 or more, about 40 or more, about 50 or more, about 75 or more, about 100 or more, about 125 or more, about 150 or more, about 200 or more, or about 250 or more in a tumor tissue section in an MT1-MMP IHC staining assay, and administering to the patient a therapeutically effective amount of BT1718, or a pharmaceutically acceptable salt thereof, or a pharmaceutical composition thereof.

60. The method of any one of claims 57-59, wherein the cancer is bladder cancer, endometrial cancer, esophageal cancer, glioblastoma, mesothelioma, multiple myeloma, ovarian cancer, pancreatic cancer, lung cancer, or prostate cancer.

61. The method of claim 60, wherein the bladder cancer is selected from the group consisting of basal, p53-like, and luminal.

62. The method of claim 60, wherein the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated.

63. The method of claim 60, wherein the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI).

64. The method of claim 60, wherein the glioblastoma is selected from the group consisting of proneural, neural, classical, and mesenchymal.

65. The method of claim 60, wherein the mesothelioma is selected from the group consisting of pleural, peritoneal, pericardial, epithelioid, sarcomatoid, biphasic, and malignant.

66. The method of claim 60, wherein the multiple myeloma is selected from the group consisting of hyperdiploid, non-hyperdiploid, cyclin D translocation, MMSET translocation, MAF translocation, and unclassified.

67. The method of claim 60, wherein the ovarian cancer is selected from the group consisting of clear cell, endometrioid, mucinous, high-grade serous and low-grade serous.

68. The method of claim 60, wherein the pancreatic cancer is selected from the group consisting of squamous, pancreatic progenitor, immunogenic, and ADEX (Aberrantly Differentiated Endocrine eXocrine).

69. The method of claim 60, wherein the lung cancer is selected from the group consisting of non-small cell lung cancer (NSCLC) and small cell lung cancer (SCLC).

70. The method of claim 60, wherein the prostate cancer is selected from the group consisting of AZGP1 (subtype I), MUC1 (subtype II), and MUC1 (subtype III).

71. The method of claim 60, wherein the bladder cancer is high MT1-MMP expressing.

72. The method of claim 60, wherein the endometrial cancer is high MT1-MMP expressing.

73. The method of claim 60, wherein the esophageal cancer is high MT1-MMP expressing.

74. The method of claim 60, wherein the glioblastoma is high MT1-MMP expressing.

75. The method of claim 60, wherein the mesothelioma is high MT1-MMP expressing.

76. The method of claim 60, wherein the multiple myeloma is high MT1-MMP expressing.

77. The method of claim 60, wherein the ovarian cancer is high MT1-MMP expressing.

78. The method of claim 60, wherein the pancreatic cancer is high MT1-MMP expressing.

79. The method of claim 60, wherein the lung cancer is high MT1-MMP expressing.

80. The method of claim 60, wherein the prostate cancer is high MT1-MMP expressing.

81. The method of claim 58 or 59, wherein the MT1-MMP IHC staining assay uses a primary MT1-MMP antibody binding to the tumor membrane (TM) or tumor stroma (TS).

82. The method of claim 81, wherein the primary MT1-MMP antibody binding to the tumor membrane (TM) is primary MT1-MMP antibody MAB3328.

83. The method of claim 59, wherein the H-score refers to an H-score for tumor membrane (TM), an H-score for tumor stroma (TS), or a composite H-score for tumor membrane (TM) and tumor stroma (TS).

84. A method of treating a cancer in a patient, comprising selecting a patient having an H-score for tumor cell membrane of about 15 or more, about 20 or more, about 30 or more, about 40 or more, about 50 or more, about 75 or more, about 100 or more, about 125 or more, or about 150 or more in a tumor tissue section in an MT1-MMP IHC staining assay, and administering to the patient in need thereof a therapeutically effective amount of BT1718, or a pharmaceutically acceptable salt thereof, or a pharmaceutical composition thereof.

85. The method of claim 84, wherein a cancer is bladder cancer, endometrial cancer, esophageal cancer, glioblastoma, mesothelioma, multiple myeloma, ovarian cancer, pancreatic cancer, lung cancer, or prostate cancer.

86. The method of claim 85, wherein the bladder cancer is selected from the group consisting of basal, p53-like, and luminal.

87. The method of claim 85, wherein the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated.

88. The method of claim 85, wherein the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI).

89. The method of claim 85, wherein the glioblastoma is selected from the group consisting of proneural, neural, classical, and mesenchymal.

90. The method of claim 85, wherein the mesothelioma is selected from the group consisting of pleural, peritoneal, pericardial, epithelioid, sarcomatoid, biphasic, and malignant.

91. The method of claim 85, wherein the multiple myeloma is selected from the group consisting of hyperdiploid, non-hyperdiploid, cyclin D translocation, MMSET translocation, MAF translocation, and unclassified.

92. The method of claim 85, wherein the ovarian cancer is selected from the group consisting of clear cell, endometrioid, mucinous, high-grade serous and low-grade serous.

93. The method of claim 85, wherein the pancreatic cancer is selected from the group consisting of squamous, pancreatic progenitor, immunogenic, and ADEX (Aberrantly Differentiated Endocrine eXocrine).

94. The method of claim 85, wherein the lung cancer is selected from the group consisting of non-small cell lung cancer (NSCLC) and small cell lung cancer (SCLC).

95. The method of claim 85, wherein the prostate cancer is selected from the group consisting of AZGP1 (subtype I), MUC1 (subtype II), and MUC1 (subtype III).

96. The method of claim 85, wherein the bladder cancer is high MT1-MMP expressing.

97. The method of claim 85, wherein the endometrial cancer is high MT1-MMP expressing.

98. The method of claim 85, wherein the esophageal cancer is high MT1-MMP expressing.

99. The method of claim 85, wherein the glioblastoma is high MT1-MMP expressing.

100. The method of claim 85, wherein the mesothelioma is high MT1-MMP expressing.

101. The method of claim 85, wherein the multiple myeloma is high MT1-MMP expressing.

102. The method of claim 85, wherein the ovarian cancer is high MT1-MMP expressing.

103. The method of claim 85, wherein the pancreatic cancer is high MT1-MMP expressing.

104. The method of claim 85, wherein the lung cancer is high MT1-MMP expressing.

105. The method of claim 85, wherein the prostate cancer is high MT1-MMP expressing.

106. The method of claim 84, wherein the MT1-MMP IHC staining assay uses a primary MT1-MMP antibody binding to tumor membrane (TM).

107. The method of claim 106, wherein the primary MT1-MMP antibody binding to the tumor membrane is primary MT1-MMP antibody MAB3328.
